SEQUENCE LISTING (90 sequences)

SEQ ID NO: 1 - protein, 20 aa, Artificial Sequence
  Description of Artificial Sequences; note = synthetic construct
PSSRASSRASSRPRPDDLEI

SEQ ID NO: 2 - protein, 9 aa, Artificial Sequence
  Description of Artificial Sequences; note = synthetic construct
RPRPDDLEI

SEQ ID NO: 3 - protein, 9 aa, Artificial Sequence
  Description of Artificial Sequences; note = synthetic construct
RPRPDDLEV

SEQ ID NO: 4 - protein, 9 aa, Artificial Sequence
  Description of Artificial Sequences; note = synthetic construct
RPRPDDVPV

SEQ ID NO: 5 - protein, 9 aa, Artificial Sequence
  Description of Artificial Sequences; note = synthetic construct
KARSDDLSV

SEQ ID NO: 6 - DNA, 27 bp, Artificial Sequence
  Description of Artificial Sequences; note = synthetic construct
agacctcggcctgatgacctggagatt

SEQ ID NO: 7 - protein, 16 aa, Artificial Sequence
  Description of Artificial Sequences; note = synthetic construct
RQPKIWFPNRRKPWKK

SEQ ID NO: 8 - protein, 36 aa, Artificial Sequence
  Description of Artificial Sequences; note = synthetic construct
RQPKIWFPNRRKPWKKPSSRASSRASSRPRPDDLEI

SEQ ID NO: 9 - protein, 25 aa, Artificial Sequence
  Description of Artificial Sequences; note = synthetic construct
RQPKIWFPNRRKPWKKRPRPDDLEI

SEQ ID NO: 10 - protein, 25 aa, Artificial Sequence
  Description of Artificial Sequences; note = synthetic construct
RQPKIWFPNRRKPWKKRPRPDDLEV

SEQ ID NO: 11 - protein, 25 aa, Artificial Sequence
  Description of Artificial Sequences; note = synthetic construct
RQPKIWFPNRRKPWKKRPRPDDVPV

SEQ ID NO: 12 - protein, 25 aa, Artificial Sequence
  Description of Artificial Sequences; note = synthetic construct
RQPKIWFPNRRKPWKKKARSDDLSV

SEQ ID NO: 13 - DNA, 74 bp, Artificial Sequence
  Description of Artificial Sequences; note = synthetic construct
cggcagcccaagatctggttccccaaccggcggaagccctggaagaagcggcccggcccgacgacctggagatc

SEQ ID NO: 14 - protein, 11 aa, Artificial Sequence
  Description of Artificial Sequences; note = synthetic construct
GRKKRRQRPPQ

SEQ ID NO: 15 - protein, 16 aa, Artificial Sequence
  Description of Artificial Sequences; note = synthetic construct
RQIKIWFQNRRMKWKK

SEQ ID NO: 16 - protein, 16 aa, Artificial Sequence
  Description of Artificial Sequences; note = synthetic construct
RQIAIWFQNRRMKWAA

SEQ ID NO: 17 - protein, 9 aa, Artificial Sequence
  Description of Artificial Sequences; note = synthetic construct
RKKRRQRRR

SEQ ID NO: 18 - protein, 21 aa, Artificial Sequence
  Description of Artificial Sequences; note = synthetic construct
TRSSRAGLQFPVGRVHRLLRK

SEQ ID NO: 19 - protein, 26 aa, Artificial Sequence
  Description of Artificial Sequences; note = synthetic construct
GWTLNSAGYLLGKINKALAALAKKIL

SEQ ID NO: 20 - protein, 18 aa, Artificial Sequence
  Description of Artificial Sequences; note = synthetic construct
KLALKLALKALKAALKLA

SEQ ID NO: 21 - protein, 16 aa, Artificial Sequence
  Description of Artificial Sequences; note = synthetic construct
AAVALLPAVLLALLAP

SEQ ID NO: 22 - protein, 10 aa, Artificial Sequence
  Description of Artificial Sequences; note = synthetic construct
VPMLKPMLKE

SEQ ID NO: 23 - protein, 28 aa, Artificial Sequence
  Description of Artificial Sequences; note = synthetic construct
MANLGYWLLALFVTMWTDVGLCKKRPKP

SEQ ID NO: 24 - protein, 18 aa, Artificial Sequence
  Description of Artificial Sequences; note = synthetic construct
LLIILRRRIRKQAHAHSK

SEQ ID NO: 25 - protein, 21 aa, Artificial Sequence
  Description of Artificial Sequences; note = synthetic construct
KETWWETWWTEWSQPKKKRKV

SEQ ID NO: 26 - protein, 18 aa, Artificial Sequence
  Description of Artificial Sequences; note = synthetic construct
RGGRLSYSRRRFSTSTGR

SEQ ID NO: 27 - protein, 15 aa, Artificial Sequence
  Description of Artificial Sequences; note = synthetic construct
SDLWEMMMVSLACQY

SEQ ID NO: 28 - protein, 12 aa, Artificial Sequence
  Description of Artificial Sequences; note = synthetic construct
TSPLNIHNGQKL

SEQ ID NO: 29 - protein, 19 aa, Artificial Sequence
  Description of Artificial Sequences; note = synthetic construct
PSRASSRASSRPRPDDLEI

SEQ ID NO: 30 - protein, 20 aa, Artificial Sequence
  Description of Artificial Sequences; note = synthetic construct
GSNKSTASSKSPDPKNSVWI

SEQ ID NO: 31 - protein, 20 aa, Artificial Sequence
  Description of Artificial Sequences; note = synthetic construct
GSNKSSASSKSGDGKNSVWI

SEQ ID NO: 32 - protein, 21 aa, Artificial Sequence
  Description of Artificial Sequences; note = synthetic construct
GRASKASRASSGRARPEDLAI

SEQ ID NO: 33 - protein, 13 aa, Artificial Sequence
  Description of Artificial Sequences; note = synthetic construct
GSASSRDGKTVWI

SEQ ID NO: 34 - protein, 19 aa, Artificial Sequence
  Description of Artificial Sequences; note = synthetic construct
PRVSVPNFGRTQSSDSAYV

SEQ ID NO: 35 - protein, 19 aa, Artificial Sequence
  Description of Artificial Sequences; note = synthetic construct
PRMSMPNFGRTQSSDSAYV

SEQ ID NO: 36 - protein, 21 aa, Artificial Sequence
  Description of Artificial Sequences; note = synthetic construct
PRAGSEKGSASSRDGKTTVWI

SEQ ID NO: 37 - protein, 23 aa, Artificial Sequence
  Description of Artificial Sequences; note = synthetic construct
GYHSDKRRLSKASSKARSDDLSV

SEQ ID NO: 38 - protein, 19 aa, Artificial Sequence
  Description of Artificial Sequences; note = synthetic construct
PLSRLSKASSRARSDDLTV

SEQ ID NO: 39 - protein, 22 aa, Artificial Sequence
  Description of Artificial Sequences; note = synthetic construct
PNHVVSLTNNLIGRRVPTDLQI

SEQ ID NO: 40 - protein, 29 aa, Artificial Sequence
  Description of Artificial Sequences; note = synthetic construct
PSCVSSSAVLTTICSSDQVVPVGLSSFYM

SEQ ID NO: 41 - protein, 21 aa, Artificial Sequence
  Description of Artificial Sequences; note = synthetic construct
GRSSKASKSSGGRARAADLAI

SEQ ID NO: 42 - protein, 17 aa, Artificial Sequence
  Description of Artificial Sequences; note = synthetic construct
LCYLLIRYCSGKSKKPV

SEQ ID NO: 43 - protein, 18 aa, Artificial Sequence
  Description of Artificial Sequences; note = synthetic construct
GQKPPSRPSSSASKKQYV

SEQ ID NO: 44 - protein, 19 aa, Artificial Sequence
  Description of Artificial Sequences; note = synthetic construct
SSRASSRASSRPRPDDLEV

SEQ ID NO: 45 - protein, 9 aa, Artificial Sequence
  Description of Artificial Sequences; note = synthetic construct
RPKPDDLEI

SEQ ID NO: 46 - protein, 19 aa, Artificial Sequence
  Description of Artificial Sequences; note = synthetic construct
SSRASSRASSRPKPDDLEI

SEQ ID NO: 47 - protein, 9 aa, Artificial Sequence
  Description of Artificial Sequences; note = synthetic construct
RPKPDDLDI

SEQ ID NO: 48 - protein, 19 aa, Artificial Sequence
  Description of Artificial Sequences; note = synthetic construct
SSRASSRASSRPRPDDLDI

SEQ ID NO: 49 - protein, 19 aa, Artificial Sequence
  Description of Artificial Sequences; note = synthetic construct
SSRASTRASSRPRPDDLEI

SEQ ID NO: 50 - protein, 9 aa, Artificial Sequence
  Description of Artificial Sequences; note = synthetic construct
RPRPEDLEI

SEQ ID NO: 51 - protein, 19 aa, Artificial Sequence
  Description of Artificial Sequences; note = synthetic construct
SSRASSRASSRPRPEDLEI

SEQ ID NO: 52 - protein, 9 aa, Artificial Sequence
  Description of Artificial Sequences; note = synthetic construct
GDGKNSVWV

SEQ ID NO: 53 - protein, 23 aa, Artificial Sequence
  Description of Artificial Sequences; note = synthetic construct
SKAGSNKSTASSKSGDGKNSVWV

SEQ ID NO: 54 - protein, 18 aa, Artificial Sequence
  Description of Artificial Sequences; note = synthetic construct
GQKPPSRPSSSASKKLYV

SEQ ID NO: 55 - protein, 24 aa, Artificial Sequence
  Description of Artificial Sequences; note = synthetic construct
RQPKIWFPNRRKPWKIELDDPRPR

SEQ ID NO: 56 - protein, 20 aa, Artificial Sequence
  Description of Artificial Sequences; note = synthetic construct
GRKKRRQRPPQRPRPDDLEI

SEQ ID NO: 57 - protein, 25 aa, Artificial Sequence
  Description of Artificial Sequences; note = synthetic construct
RQIKIWFQNRRMKWKKRPRPDDLEI

SEQ ID NO: 58 - protein, 25 aa, Artificial Sequence
  Description of Artificial Sequences; note = synthetic construct
RQIAIWFQNRRMKWAARPRPDDLEI

SEQ ID NO: 59 - protein, 18 aa, Artificial Sequence
  Description of Artificial Sequences; note = synthetic construct
RKKRRQRRRRPRPDDLEI

SEQ ID NO: 60 - protein, 30 aa, Artificial Sequence
  Description of Artificial Sequences; note = synthetic construct
TRSSRAGLQFPVGRVHRLLRKRPRPDDLEI

SEQ ID NO: 61 - protein, 35 aa, Artificial Sequence
  Description of Artificial Sequences; note = synthetic construct
GWTLNSAGYLLGKINKALAALAKKILRPRPDDLEI

SEQ ID NO: 62 - protein, 27 aa, Artificial Sequence
  Description of Artificial Sequences; note = synthetic construct
KLALKLALKALKAALKLARPRPDDLEI

SEQ ID NO: 63 - protein, 25 aa, Artificial Sequence
  Description of Artificial Sequences; note = synthetic construct
AAVALLPAVLLALLAPRPRPDDLEI

SEQ ID NO: 64 - protein, 19 aa, Artificial Sequence
  Description of Artificial Sequences; note = synthetic construct
VPMLKPMLKERPRPDDLEI

SEQ ID NO: 65 - protein, 37 aa, Artificial Sequence
  Description of Artificial Sequences; note = synthetic construct
MANLGYWLLALFVTMWTDVGLCKKRPKPRPRPDDLEI

SEQ ID NO: 66 - protein, 27 aa, Artificial Sequence
  Description of Artificial Sequences; note = synthetic construct
LLIILRRRIRKQAHAHSKRPRPDDLEI

SEQ ID NO: 67 - protein, 30 aa, Artificial Sequence
  Description of Artificial Sequences; note = synthetic construct
KETWWETWWTEWSQPKKKRKVRPRPDDLEI

SEQ ID NO: 68 - protein, 27 aa, Artificial Sequence
  Description of Artificial Sequences; note = synthetic construct
RGGRLSYSRRRFSTSTGRRPRPDDLEI

SEQ ID NO: 69 - protein, 24 aa, Artificial Sequence
  Description of Artificial Sequences; note = synthetic construct
SDLWEMMMVSLACQYRPRPDDLEI

SEQ ID NO: 70 - protein, 21 aa, Artificial Sequence
  Description of Artificial Sequences; note = synthetic construct
TSPLNIHNGQKLRPRPDDLEI

SEQ ID NO: 71 - protein, 120 aa, Artificial Sequence
  Description of Artificial Sequences; note = synthetic construct
KGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVD

SEQ ID NO: 72 - protein, 120 aa, Artificial Sequence
  Description of Artificial Sequences; note = synthetic construct
KTDPYSHSGTMSPSKDCGSPKYAYYNGCSSPTAPLSPMSPPGYKLVTGDRNNSSCRNYNKQASEQNWANYSAEQNRMGQAGSTISNSHAQPFDFADEHQNTKKLASGHELQPLTIVDQRP

SEQ ID NO: 73 - protein, 120 aa, Artificial Sequence
  Description of Artificial Sequences; note = synthetic construct
LGFGTIRDSLNSKRRELEDPGAYNYPFTWNTPSAPPGYNIAVKPDQIQYTELSNAKIAYKQNKANTAQEQQYGSHEENLPADLEALQREIRMAQERLDLAVQAYSHQNNPHGPREKKAKV

SEQ ID NO: 74 - protein, 120 aa, Artificial Sequence
  Description of Artificial Sequences; note = synthetic construct
GFGTIRDTLNNKRKELEDSGTYNYPFTWNTPSAPPGYNIAVKPDQMQYTELSNAKMAYKQNKANIAQEQQYGSNEENIPADLENLQREIKVAQERLDMAIQAYNNQNNPGSSSREKKSKA

SEQ ID NO: 75 - protein, 120 aa, Artificial Sequence
  Description of Artificial Sequences; note = synthetic construct
PYLVDCFVSRPTEKTIFIIFMLVVGLISLVLNLLELVHLLCRCLSRGMRARQGQDAPPTQGTSSDPYTDQVFFYLPVGQGPSSPPCPTYNGLSSSEQNWANLTTEERLASSRPPLFLDPP

SEQ ID NO: 76 - protein, 120 aa, Artificial Sequence
  Description of Artificial Sequences; note = synthetic construct
CGSKEHGNRKMRGRLLLTYMASIFFKSVFEVAFLLIQWYLYGFTLSAVYICEQSPCPHRVDCFLSRPTEKTIFILFMLVVSMVSFVLNVIELFYVLFKAIKNHLGNEKEEVYCNPVELQK

SEQ ID NO: 77 - protein, 239 aa, Artificial Sequence
  Description of Artificial Sequences; note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 78 - DNA, 60 bp, Artificial Sequence
  Description of Artificial Sequences; note = synthetic construct
ccctcctcccgggcctcctcccgggcctcctcccggccccggcccgacgacctggagatc

SEQ ID NO: 79 - DNA, 27 bp, Artificial Sequence
  Description of Artificial Sequences; note = synthetic construct
cggccccggcccgacgacctggagatc

SEQ ID NO: 80 - DNA, 27 bp, Artificial Sequence
  Description of Artificial Sequences; note = synthetic construct
cggccccggcccgacgacctggaggtg

SEQ ID NO: 81 - DNA, 27 bp, Artificial Sequence
  Description of Artificial Sequences; note = synthetic construct
cggccccggcccgacgacgtgcccgtg

SEQ ID NO: 82 - DNA, 27 bp, Artificial Sequence
  Description of Artificial Sequences; note = synthetic construct
aaggcccggtccgacgacctgtccgtg

SEQ ID NO: 83 - DNA, 48 bp, Artificial Sequence
  Description of Artificial Sequences; note = synthetic construct
cggcagcccaagatctggttccccaaccggcggaagccctggaagaag

SEQ ID NO: 84 - DNA, 108 bp, Artificial Sequence
  Description of Artificial Sequences; note = synthetic construct
cggcagcccaagatctggttccccaaccggcggaagccctggaagaagccctcctcccgggcctcctcccgggcctcctcccggccccggcccgacgacctggagatc

SEQ ID NO: 85 - DNA, 75 bp, Artificial Sequence
  Description of Artificial Sequences; note = synthetic construct
cggcagcccaagatctggttccccaaccggcggaagccctggaagaagcggccccggcccgacgacctggagatc

SEQ ID NO: 86 - DNA, 75 bp, Artificial Sequence
  Description of Artificial Sequences; note = synthetic construct
cggcagcccaagatctggttccccaaccggcggaagccctggaagaagcggccccggcccgacgacctggaggtg

SEQ ID NO: 87 - DNA, 75 bp, Artificial Sequence
  Description of Artificial Sequences; note = synthetic construct
cggcagcccaagatctggttccccaaccggcggaagccctggaagaagcggccccggcccgacgacgtgcccgtg

SEQ ID NO: 88 - DNA, 75 bp, Artificial Sequence
  Description of Artificial Sequences; note = synthetic construct
cggcagcccaagatctggttccccaaccggcggaagccctggaagaagaaggcccggtccgacgacctgtccgtg

SEQ ID NO: 89 - protein, 20 aa, Artificial Sequence
  Description of Artificial Sequences; note = synthetic construct
PCSRASSRMSSRARPDDLDV

SEQ ID NO: 90 - protein, 19 aa, Artificial Sequence
  Description of Artificial Sequences; note = synthetic construct
PRVSVPNFGRTQSSDSAYV

(19) United States
(12) Patent Application Publication
(10) Pub. No.: US 2021/0007347 A1
(43) Pub. Date: Jan. 14, 2021

(54) COMPOSITIONS AND METHODS FOR PRESERVING ORGAN TRANSPLANTS

(71) Applicant: FIRSTSTRING RESEARCH, INC., Mt. Pleasant, SC (US)

(72) Inventor: Gautam S. GHATNEKAR, Mt. Pleasant, SC (US)

(21) Appl. No.: 16/923,289

(22) Filed: Jul. 8, 2020

Related U.S. Application Data
(60) Provisional application No. 62/871,475, filed on Jul. 8, 2019.

(51) Int. Cl. A01N 1/02; C07K 14/435

(52) U.S. Cl. CPC A01N 1/0221; C07K 14/435

(57) ABSTRACT

The present disclosure provides compositions and methods for preserving organs and tissues for transplantation, and for preventing cellular injury in organs or in subjects.

### CROSS REFERENCE TO RELATED APPLICATIONS

This application claims the benefit of priority to U.S. Provisional Application No. 62/871,475, filed on Jul. 8, 2019, which is hereby incorporated by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: FIRS_010_01US_SeqList_ST25.txt, date recorded: Jul. 8, 2020, file size 34 kilobytes).

### BACKGROUND

Organ or tissue transplant has become an established technique for treatment of various diseases and disorders. Primary organs that can be transplanted include kidney, liver, heart, lungs, pancreas, and intestine. Tissues that can be transplanted include bones, tendons, corneae, skin, heart valves, nerves, and veins. The decrease in viability of the organ or tissue after removal from the donor is a significant limiting factor to the success of organ and tissue transplants. Generally, organs and tissues are preserved after removal from the donor by hypothermic storage and/or continuous perfusion. Hypothermic storage, or cold storage, generally means rapid cooling of the organ or tissue to a temperature between 0° and 4° C. Hypothermic storage and perfusion are performed to decreases the rate at which intracellular enzymes degrade. Nevertheless, injury to the organ occurs through damage to epithelial and endothelial cells, during cold storage and upon reperfusion with a warm reperfusion solution upon transplant into the recipient. Such ischemia reperfusion injury to organs commonly leads to delayed or diminished organ or tissue function, and predisposes the organ or tissue to rejection. Moreover, the number of patients waiting for transplantation greatly exceeds the number of available donor organs and tissues, and organs and tissues collected for transplant often become unsuitable for transplantation and/or fail after transplantation due to injury caused by brain death of the donor and complications relating to prolonged storage, as well as ischemia reperfusion injury. Current preservation solutions used in hospitals contribute little to improving post-transplant outcomes and do not address the molecular/cellular features contributing to vasculopathy and immune rejection that determine graft function and survival.

Therefore, there is a need in the art for improved methods of preservation of organs and tissues, to extend the viability of the organ or tissue and to improve organ or tissue function following transplant.

### BRIEF SUMMARY

In accordance with the purpose of this invention, as embodied and broadly described herein, this invention relates to methods and compositions for preserving organs or tissues for organ or tissue transplantation.

In one aspect, the present disclosure provides methods and compositions for preserving organs or tissues for organ transplantation or tissue transplantation, comprising contacting the organ or tissue with a solution comprising an isolated polypeptide comprising the carboxy-terminal amino acid sequence of an alpha connexin, or a conservative variant thereof. In some embodiments, contacting the organ or tissue with the solution comprises incubating the organ or tissue with the solution. In further embodiments, the incubation is during cold storage of the organ or tissue prior to transplantation of the organ or tissue. In some embodiments, contacting the organ or tissue comprises perfusing the organ or tissue with the solution following removal from the donor. In some embodiments, contacting the organ or tissue comprises incubating the organ or tissue with the solution and perfusing the organ or tissue with the solution. In some embodiments, contacting the organ or tissue with the solution comprises administering the solution to an organ or tissue donor prior to organ or tissue removal. In some embodiments, the organ is selected from the group consisting of a kidney, heart, liver, lung, pancreas, thymus, and intestine.

In some embodiments, the present disclosure provides methods for treating a subject in need of an organ or tissue transplant. In further embodiments, the methods comprise preserving the organ or tissue in a solution comprising an alpha connexin polypeptide provided herein. In some embodiments, the methods comprise inhibiting cellular injury in the organs or tissues. In some embodiments, the polypeptide inhibits cellular injury in the organs. In one embodiment, the polypeptide inhibits endothelial cellular injury. In another embodiment, the polypeptide inhibits epithelial cellular injury. In one aspect, the cellular injury is caused by cold preservation induced damage. In another aspect, the cellular injury is caused by hypoxia.

In one aspect, the cellular injury is ischemia reperfusion injury (IRI). In another aspect, the cellular injury is ischemic reperfusion induced graft injury.

In one embodiment, the polypeptide promotes cell-cell communication. In another embodiment, the polypeptide stabilizes gap junctions in cells. In yet another embodiment, the polypeptide stabilizes tight junctions in cells. In one embodiment, the polypeptide mitigates hemichannel activity in cells. In one embodiment, the polypeptide inhibits apoptosis in cells. In another embodiment, the polypeptide inhibits mitochondrial oxidant production. In another embodiment, the polypeptide promotes the integrity of endothelial cells. In another embodiment, the polypeptide promotes barrier function of endothelial cells. In some embodiments, the cells are the cells of an organ for transplantation from a donor to a recipient.

In one embodiment, the polypeptide inhibits post transplantation IRI by inhibiting post transplantation inflammation. In one embodiment, the polypeptide preserves organs by inhibiting pro-inflammatory cytokine release from cells in the organs. In one aspect, the pro-inflammatory cytokine is IL-8.

In one embodiment, the polypeptide comprises the amino acid sequence selected from the group consisting of SEQ ID NO: 1, SEQ ID NO: 2, SEQ ID NO: 3, SEQ ID NO: 4, and SEQ ID NO: 5. In another embodiment, the polypeptide comprises the amino acid sequence of SEQ ID NO: 2. In another embodiment, the polypeptide comprises an amino acid sequence with at least 65% sequence identity to the c-terminal most 9 amino acids of SEQ ID NO: 1. In some embodiments, the polypeptide comprises from about 4 to about 30 contiguous amino acids of the carboxy-terminus of the alpha connexin. In some embodiments, the polypeptide comprises from about 5 to about 19 contiguous amino acids of the carboxy-terminus of the alpha connexin. In some embodiments, the polypeptide comprises a deletion of one amino acid from the carboxy-terminal amino acid sequence.

In one embodiment, the alpha connexin is selected from a group consisting of connexin 30.2, connexin 31.9, connexin 33, connexin 35, connexin 36, connexin 37, connexin 38, connexin 39, connexin 39.9, connexin 40, connexin 40.1, connexin 43, connexin 43.4, connexin 44, connexin 44.2, connexin 44.1, connexin 45, connexin 46, connexin 46.6, connexin 47, connexin 49, connexin 50, connexin 56, and connexin 59. In another embodiment, the alpha connexin is connexin 37, connexin 40, connexin 43, or connexin 45. In some embodiments, the alpha connexin is connexin 43. In some embodiments, the alpha connexin

In one aspect, the isolated peptides provided herein comprise an alpha connexin polypeptide and a cellular penetration sequence. In some embodiments, the cellular penetration sequence is an antennapedia sequence. In some embodiments, the isolated peptide comprises SEQ ID NO: 9. The peptide comprising SEQ ID NO: 9 is referred to herein, in some embodiments, as ACT1.

In some embodiments, the polypeptide is present in the solution at a concentration of less than about 50 μM, less than about 40 μM, less than about 30 μM, less than about 20 μM, less than about 10 μM, less than about 5 μM, less than about 2 μM, or less than about 1 μM. In some embodiments, the polypeptide is present in the solution at a concentration of between about 1 μM to about 10 μM. In some embodiments, the polypeptide is present in the solution at a concentration of about 10 μM, about 9 μM, about 8 μM, about 7 μM, about 6 μM, about 5 μM, about 4 μM, about 3 μM, about 2 μM, or about 1 μM.

Thus, in some embodiments, the present disclosure provides methods and compositions for preserving an organ or tissue for transplantation, the method comprising contacting the organ or tissue with ACT1 peptide, wherein ACT1 peptide is present in an amount of less than about 10 μM. In some embodiments, the present disclosure provides methods and compositions for preserving an organ or tissue for transplantation, the method comprising contacting the organ or tissue with ACT1 peptide, wherein ACT1 peptide is present in an amount of about 1 μM.

In one embodiment, the present disclosure provides methods and compositions for inhibiting cellular injury in a subject, comprising administering to the subject an isolated polypeptide comprising the carboxy-terminal amino acid sequence of an alpha connexin, or a conservative variant thereof. In one embodiment, the cellular injury is an endothelial cellular injury. In another embodiment, the cellular injury is an epithelial cellular injury. In one embodiment, the cellular injury is a post transplantation IRI. In one aspect, the polypeptide inhibits post transplantation IRI by inhibiting endothelial permeability. In another aspect, the polypeptide inhibits post transplantation IRI by inhibiting heart graft injury.

In one aspect, the present disclosure provides compositions comprising an organ preservation solution and a polypeptide comprising the carboxy-terminus of an alpha connexin provided herein. In another aspect, the present disclosure provides compositions comprising one or more organs for transplant and a polypeptide comprising the carboxy-terminus of an alpha connexin, or a conservative variant thereof, as provided herein. In some embodiments, the polypeptide is present in the solution or composition in an amount effective for reversing cellular injury in the organ or tissue. In some embodiments, the polypeptide is present in the solution or composition in an amount less than about 10 μM. In some embodiments, the polypeptide is present in an amount of about 1 μM to about 10 μM. In some embodiments, the polypeptide is present in an amount of about 1 μM. In some embodiments, the polypeptide comprises ACT1. In some embodiments, the polypeptide comprises or consists of SEQ ID NO: 2 or SEQ ID NO: 9.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B, and 1C show that ACT1 pretreatment of endothelial cells (ECs) prevents cold storage and reperfusion injury and reduces pro-inflammatory cytokine release.

FIG. 2A is a schematic showing the allogenic heart transplantation model used in Example 2. FIGS. 2B, 2C, and 2D show that addition of ACT1 peptide to the UW cold storage solution improves organ characteristics and function.

FIGS. 3A and 3B show that ACT1 pre-treatment reduces chronic rejection of donor aortas.

FIGS. 4A, 4B, and 4C shows that ACT1 peptide improves organ characteristics in a first paired set of marginal kidneys.

FIGS. 5A, 5B, and 5C shows that ACT1 peptide improves organ characteristics in a second paired set of marginal kidneys.

FIG. 6 shows that ACT1 peptide reduces LDH activity in a paired set of marginal kidneys.

FIG. 7 shows that ACT1 peptide reduces Vcam and Icam expression in a paired set of marginal kidneys.

### DETAILED DESCRIPTION

Provided herein are compositions and methods for preserving organs and tissue for transplantation, comprising contacting the organ or tissue with a polypeptide comprising a carboxy-terminal amino acid sequence of an alpha Connexin (also referred to herein as an alpha Connexin carboxy-Terminal (ACT) polypeptide), or a conservative variant thereof. The methods advantageously improve donor organ and tissue characteristics including rescuing marginal organs and tissues that would otherwise not be suitable for transplantation or would otherwise fail or become rejected shortly after transplantation due to, for example, long storage time and/or damage to the organ or tissue prior to, during, or after organ harvest.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Provided for use in the disclosed methods is an isolated polypeptide comprising a carboxy-terminal amino acid sequence of an alpha Connexin (also referred to herein as an alpha Connexin carboxy-Terminal (ACT) polypeptide), or a conservative variant thereof. The ACT polypeptides of the provided method are disclosed in International Patent Publication WO/2006/069181 and U.S. Pat. No. 9,844,214, each of which is incorporated by reference herein in its entirety. In some aspects, the polypeptide of the disclosed methods can be any polypeptide comprising the carboxy-terminal most amino acids of an alpha Connexin. In some embodiments, the polypeptide useful in the disclosed methods is the peptide referred to herein as ACT1. ACT1 comprises an alpha connexin peptide according to SEQ ID NO: 2 and a cell penetrating peptide. In some embodiments, ACT1 comprises SEQ ID NO: 9.

In some aspects, the polypeptide does not comprise the full-length alpha Connexin protein. Thus, in some aspects, the provided polypeptide does not comprise the cytoplasmic N-terminal domain of the alpha Connexin. In some aspects, the provided polypeptide does not comprise the two extracellular domains of the alpha Connexin. In some aspects, the provided polypeptide does not comprise the four transmembrane domains of the alpha Connexin. In some aspects, the provided polypeptide does not comprise the cytoplasmic loop domain of the alpha Connexin. In some aspects, the provided polypeptide does not comprise that part of the sequence of the cytoplasmic carboxyl terminal domain of the alpha Connexin proximal to the fourth transmembrane domain. There is a conserved proline or glycine residue in alpha Connexins consistently positioned some 17 to 30 amino acids from the carboxyl terminal-most amino acid (Table 2). For example, for human Cx43 a proline residue at amino acid 363 is positioned 19 amino acids back from the carboxyl terminal most isoleucine. In another example, for chick Cx43 a praline residue at amino acid 362 is positioned 18 amino acids back from the carboxyl terminal-most isoleucine. In another example, for human Cx45 a glycine residue at amino acid 377 is positioned 19 amino acids back from the carboxyl terminal most isoleucine. In another example for rat Cx33, a praline residue at amino acid 258 is positioned 28 amino acids back from the carboxyl terminal most methionine. Thus, in some aspects, the provided polypeptide does not comprise amino acids proximal to said conserved proline or glycine residue of the alpha Connexin. Thus, the provided polypeptide can comprise the c-terminal-most 4 to 30 amino acids of the alpha Connexin, including the c-terminal most 4, 5, 6, 7, 8, 9, 10, 11, 12, 13, 14, 15, 16, 17, 18, 19, 20, 21, 22, 23, 24, 25, 26, 27, 28, 29, 30 amino acids of the alpha Connexin.

In some aspects, the provided polypeptide further comprises a deletion of one or more amino acids of the c-terminal-most 4 to 30 amino acids of the alpha Connexin, including a deletion of 1, 2, 3, 4, 5, 6, 7, 8, 9, or 10 amino acids of the c-terminal-most 4 to 30 amino acids of the alpha Connexin. For example, in some aspects, the provided polypeptide does not comprise the c-terminal-most 1, 2, or 3 amino acids of the alpha Connexin. For example, the provided polypeptide can consist essentially of the amino acid sequence SEQ ID NO:92, or a carboxy terminal fragment thereof of at least 4, 5, 6, 7, 8, 9, 10 amino acids in length.

The carboxy-terminal most amino acids of an alpha Connexin in the provided peptides can be flanked by non-alpha Connexin or non-ACT peptide Connexin amino acids. Examples of the flanking non-alpha Connexin and non-ACT Connexin amino acids are provided herein. An example of non-ACT Connexin amino acids are the carboxy-terminal 21 to 120 amino acids of human Cx43 (SEQ ID NO: 71). Another example would be the carboxy-terminal 21 to 120 amino acids of chick Cx43 (SEQ ID NO: 72). Another example would be the carboxy-terminal 20 to 120 amino acids of human Cx45 (SEQ ID NO: 73). Another example would be the carboxy-terminal 21 to 120 amino acids of chick Cx45 (SEQ ID NO: 74). Another example would be the carboxy-terminal 21 to 120 amino of human Cx37 (SEQ ID NO: 75). Another example would be the carboxy-terminal 21 to 120 amino acids of rat Cx33 (SEQ ID NO: 76). By “carboxy-terminal 21 to 120 amino acids” is meant the up to 120 c-terminal amino acids of the Connexin but not including the c-terminal-most 20 amino acids.

An example of a non-alpha Connexin is the 239 amino acid sequence of enhanced green fluorescent protein (SEQ ID NO: 77). In some aspects, given that ACT1 is shown to be functional when fused to the carboxy terminus of the 239 amino acid sequence of GFP, ACT peptides are expected to retain function when flanked with non-Connexin polypeptides of up to at least 239 amino acids. Indeed, as long as the ACT sequence is maintained as the free carboxy terminus of a given polypeptide, and the ACT peptide is able to access its targets. Thus, polypeptides exceeding 239 amino acids in addition to the ACT peptide can function in treating or preventing pathologies involving epithelial permeabilization and/or neovascularization.

Connexins are the sub-unit protein of the hemichannel and the gap junction channel, which are responsible for intercellular communication (Goodenough and Paul, 2003). Thus, various cells are able to communicate with each other and with the extracellular environment through hemichannels and gap junctions formed by the protein connexin. Six connexin proteins make up one hemichannel, and 2 hemichannels make up 1 gap junction channel. Gap junctions are a cluster of channels that are located in the plasma membrane between adjoining cells and they mediate intercellular communication. Hemichannels are a separate entity from gap junction channels. Hemichannels permit the exchange of molecules between the intracellular compartments and the extracellular environment.

Based on patterns of conservation of nucleotide sequence, the genes encoding Connexin proteins are divided into two families termed the alpha and beta Connexin genes. The carboxy-terminal-most amino acid sequences of alpha Connexins are characterized by multiple distinctive and conserved features (see Table 2). This conservation of organization is consistent with the ability of ACT peptides to form distinctive 3D structures, interact with multiple partnering proteins, mediate interactions with lipids and membranes, interact with nucleic acids including DNA, transit and/or block membrane channels and provide consensus motifs for proteolytic cleavage, protein cross-linking, ADP-ribosylation, glycosylation and phosphorylation. Thus, the provided polypeptide interacts with a domain of a protein that normally mediates the binding of said protein to the carboxy-terminus of an alpha Connexin. For example, nephroblastoma overexpressed protein (NOV) interacts with a Cx43 c-terminal domain (Fu et al., J Biol Chem. 2004 279(35):36943-50). It is considered that this and other proteins interact with the carboxy-terminus of alpha Connexins and further interact with other proteins forming a macromolecular complex. Thus, the provided polypeptide can inhibit the operation of a molecular machine, such as, for example, one involved in regulating the aggregation of Cx43 gap junction channels.

The ACT sequence of the provided polypeptide can be from any alpha Connexin. Thus, the alpha Connexin component of the provided polypeptide can be from a human, murine, bovine, monotrene, marsupial, primate, rodent, cetacean, mammalian, avian, reptilian, amphibian, piscine, chordate, protochordate or other alpha Connexin.

Thus, the provided polypeptide can comprise an ACT of a Connexin selected from the group consisting of mouse Connexin 47, human Connexin 47, Human Connexin 46.6, Cow Connexin 46.6, Mouse Connexin 30.2, Rat Connexin 30.2, Human Connexin 31.9, Dog Connexin 31.9, Sheep Connexin 44, Cow Connexin 44, Rat Connexin 33, Mouse Connexin 33, Human Connexin 36, mouse Connexin 36, rat Connexin 36, dog Connexin 36, chick Connexin 36, zebrafish Connexin 36, morone Connexin 35, morone Connexin 35, Cynops Connexin 35, Tetraodon Connexin 36, human Connexin 37, chimp Connexin 37, dog Connexin 37, Cricetulus Connexin 37, Mouse Connexin 37, Mesocricetus Connexin 37, Rat Connexin 37, mouse Connexin 39, rat Connexin 39, human Connexin 40.1, Xenopus Connexin 38, Zebrafish Connexin 39.9, Human Connexin 40, Chimp Connexin 40, dog Connexin 40, cow Connexin 40, mouse Connexin 40, rat Connexin 40, Cricetulus Connexin 40, Chick Connexin 40, human Cormexin 43, Cercopithecus Connexin 43, Oryctolagus Cormexin 43, Spermophilus Connexin 43, Cricetulus Connexin 43, Phodopus Connexin 43, Rat Connexin 43, Sus Connexin 43, Mesocricetus Connexin 43, Mouse Connexin 43, Cavia Connexin 43, Cow Connexin 43, Erinaceus Connexin 43, Chick Connexin 43, Xenopus Connexin 43, Oryctolagus Connexin 43, Cyprinus Connexin 43, Zebrafish Connexin 43, Danio aequipinnatus Connexin 43, Zebrafish Connexin 43.4, Zebrafish Connexin 44.2, Zebrafish Connexin 44.1, human Connexin 45, chimp Connexin 45, dog Connexin 45, mouse Connexin 45, cow Connexin 45, rat Connexin 45, chick Connexin 45, Tetraodon Connexin 45, chick Connexin 45, human Connexin 46, chimp Connexin 46, mouse Connexin 46, dog Connexin 46, rat Connexin 46, Mesocricetus Connexin 46, Cricetulus Connexin 46, Chick Connexin 56, Zebrafish Connexin 39.9, cow Connexin 49, human Connexin 50, chimp Connexin 50, rat Connexin 50, mouse Connexin 50, dog Connexin 50, sheep Connexin 49, Mesocricetus Connexin 50, Cricetulus Connexin 50, Chick Connexin 50, human Connexin 59, or other alpha Connexin. Amino acid sequences for alpha connexins are known in the art and include those identified in Table 1 by accession number.

TABLE 1
Alpha Connexins

| Protein | Accession No. | Protein | Accession No. |
|---|---|---|---|
| mouse Connexin 47 | NP_536702 | Phodopus Connexin 43 | AAR33085 |
| human Connexin 47 | AAH89439 | Rat Connexin 43 | AAH81842 |
| Human Connexin46.6 | AAB94511 | Sus Connexin 43 | AAR33087 |
| Cow Connexin 46.6 | XP_582393 | Mesocricetus Connexin 43 | AAO61857 |
| Mouse Connexin 30.2 | NP_848711 | Mouse Connexin 43 | AAH55375 |
| Rat Connexin 30.2 | XP_343966 | Cavia Connexin 43 | AAU06305 |
| Human Connexin 31.9 | AAM18801 | Cow Connexin 43 | NP_776493 |
| Dog Connexin 31.9 | XP_548134 | Erinaceus Connexin 43 | AAR33083 |
| Sheep Connexin 44 | AAD56220 | Chick Connexin 43 | AAA53027 |
| Cow Connexin 44 | I46053 | Xenopus Connexin 43 | NP_988856 |
| Rat Connexin 33 | P28233 | Oryctolagus Connexin 43 | AAS89649 |
| Mouse Connexin 33 | AAR28037 | Cyprinus Connexin 43 | AAG17938 |
| Human Connexin 36 | Q9UKL4 | Zebrafish Connexin 43 | CAH69066 |
| mouse Connexin 36 | NP_034420 | Danio aequipinnatus Connexin 43 | AAC19098 |
| rat Connexin 36 | NP_062154 | Zebrafish Connexin 43.4 | NP_571144 |
| dog Connexin 36 | XP_544602 | Zebrafish Connexin 44.2 | AAH45279 |
| chick Connexin 36 | NP_989913 | Zebrafish Connexin 44.1 | NP_571884 |
| zebrafish Connexin 36 | NP_919401 | human Connexin45 | I38430 |
| morone Connexin 35 | AAC31884 | chimp Connexin45 | XP_511557 |
| morone Connexin 35 | AAC31885 | dog Connexin 45 | XP_548059 |
| Cynops Connexin 35 | BAC22077 | mouse Connexin 45 | AAH71230 |
| Tetraodon Connexin 36 | CAG06428 | cow Connexin 45 | XP_588395 |
| human Connexin 37 | I55593 | rat Connexin 45 | AAN17802 |
| chimp Connexin 37 | XP_524658 | chick Connexin45 | NP_990834 |
| dog Connexin 37 | XP_539602 | Tetraodon Connexin 45 | CAF93782 |
| Cricetulus Connexin 37 | AAR98615 | chick Connexin 45.6 | I50219 |
| Mouse Connexin 37 | AAH56613 | human Connexin 46 | NP_068773 |
| Mesocricetus Connexin37 | AAS83433 | chimp Connexin 46 | XP_522616 |
| Rat Connexin37 | AAH86576 | mouse Connexin 46 | NP_058671 |
| mouse Connexin 39 | NP_694726 | dog Connexin 46 | XP_543178 |
| rat Connexin 39 | AAN17801 | rat Connexin 46 | NP_077352 |
| human Connexin 40.1 | NP_699199 | Mesocricetus Connexin 46 | AAS83437 |
| Xenopus Connexin38 | AAH73347 | Cricetulus Connexin 46 | AAS77618 |
| Zebrafish Connexin 39.9 | NP_997991 | Chick Connexin 56 | A45338 |
| Human Connexin 40 | NP_859054 | Zebrafish Connexin 39.9 | NP_997991 |
| Chimp Connexin 40 | XP_513754 | cow Connexin 49 | XP_602360 |
| dog Connexin 40 | XP_540273 | human Connexin 50 | P48165 |
| cow Connexin 40 | XP_587676 | chimp Connexin 50 | XP_524857 |
| mouse Connexin 40 | AAH53054 | rat Connexin 50 | NP_703195 |
| rat Connexin 40 | AAH70935 | mouse Connexin 50 | AAG59880 |
| Cricetulus Connexin 40 | AAP37454 | dog Connexin 50 | XP_540274 |
| Chick Connexin 40 | NP_990835 | sheep Connexin 49 | AAF01367 |
| human Connexin 43 | P17302 | Mesocricetus Connexin 50 | AAS83438 |
| Cercopithecus Connexin 43 | AAR33082 | Cricetulus Connexin 50 | AAR98618 |
| Oryctolagus Connexin 43 | AAR33084 | Chick Connexin 50 | BAA05381 |
| Spermophilus Connexin 43 | AAR33086 | human Connexin 59 | AAG09406 |
| Cricetulus Connexin 43 | AA061858 | | |

Thus, the provided polypeptide can comprise the amino acid sequence SEQ ID NO:1, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:43, SEQ ID NO:90, SEQ ID NO:91, or SEQ ID NO:92 or conservative variants or fragments thereof.

The 20-30 carboxy-terminal-most amino acid sequence of alpha Connexins are characterized by a distinctive and conserved organization. This distinctive and conserved organization would include a type II PDZ binding motif (Φ-x-Φ); wherein x=any amino acid and Φ=a Hydrophobic amino acid; e.g., Table 2, BOLD) and proximal to this motif, Praline (P) and/or Glycine (G) hinge residues; a high frequency phospho-Serine (S) and/or phospho-Threonine (T) residues; and a high frequency of positively charged Arginine (R), Lysine (K) and negatively charged Aspartic acid (D) or Glutamic acid (E) amino acids. For many alpha Connexins, the P and G residues occur in clustered motifs (e.g., Table 2, italicized) proximal to the carboxy-terminal type II PDZ binding motif. The S and T phosphor-amino acids of most alpha Connexins also are typically organized in clustered, repeat-like motifs (e.g., Table 2, underlined). This organization is particularly the case for Cx43, where 90% of 20 carboxyl terminal-most amino acids are comprised of the latter seven amino acids. In a further example of the high conservation of the sequence, ACT peptide organization of Cx43 is highly conserved from humans to fish (e.g., compare Cx43 ACT sequences for humans and zebrafish in Table 2). In another example, the ACT peptide organization of Cx45 is highly conserved from humans to birds (e.g., compare Cx45 ACT sequences for humans and chick in Table 2)). In another example, the ACT peptide organization of Cx36 is highly conserved from primates to fish (e.g., compare Cx36 ACT sequences for chimp and zebrafish in Table 2).

TABLE 2
Alpha Connexin Carboxy-Terminal (ACT) Amino Acid Sequences

| Gene | Sequence | SEQ ID NO |
|---|---|---|
| Human alpha Cx43 | P SSRA SSR PRP D DLEI | (SEQ ID NO: 1) |
| Chick alpha Cx43 | P S RA SSRA SSR PRP D DLEI | (SEQ ID NO: 29) |
| Zebrafish alpha Cx43 | P CSRA SSRM SSRA R P D DLDV | (SEQ ID NO: 89) |
| Human alpha Cx45 | G SNKS TA SSKS GDG KN SVWI | (SEQ ID NO: 30) |
| Chick alpha Cx45 | G SNKSS A SSKS GDG KN SVWI | (SEQ ID NO: 31) |
| Human alpha Cx46 | G RA SKAS RASS [symbol] RAR [symbol] E DLAI | SEQ ID NO: 32) |
| Human alpha Cx46.6 | G SASS RD [symbol] K TVWI | (SEQ ID NO: 33) |
| Chimp alpha Cx36 | P RVSV PNFG R TQ SSD SAYV | (SEQ ID NO: 34) |
| Chick alpha Cx36 | P RMSM PNFG R TQ SSD S AYV | (SEQ ID NO: 35) |
| Zebrafish alpha Cx36 | P RMSM PNFG R TQ SSD S AYV | (SEQ ID NO: 90) |
| Human alpha Cx47 | P RAGSEK G SASS R DG KT TVWI | (SEQ ID NO: 36) |
| Human alpha Cx40 | G HRL [symbol] YHSDKRRL SKASS KARSD DLSV | (SEQ ID NO: 37) |
| Human alpha Cx50 | P ELTTDDAR P LSRL SKASS RARSD DLTV | (SEQ ID NO: 38) |
| Human alpha Cx59 | P NHVV SLTN NLI GRRVP T DLQI | (SEQ ID NO: 39) |
| Rat alpha Cx33 | P S CV SSS A VLTTIC SS DQVV PVG L SS FYM | (SEQ ID NO: 40) |
| Sheep alpha Cx44 | G R SSKA SKSS GG RARAA DLAI | (SEQ ID NO: 41) |
| Human beta Cx26 | LC YLLIR YCSGK SKKPV | (SEQ ID NO: 42) |

Thus, in some aspects, the provided polypeptide comprises one, two, three or all of the amino acid motifs selected from the group consisting of 1) a type II PDZ binding motif, 2) Proline (P) and/or Glycine (G) hinge residues; 3) clusters of phospho-Serine (S) and/or phospho-Threonine (T) residues; and 4) a high frequency of positively charged Arginine (R) and Lysine (K) and negatively charged Aspartic acid (D) and/or Glutamic acid (E) amino acids). In some aspects, the provided polypeptide comprises a type II PDZ binding motif at the carboxy-terminus, Praline (P) and/or Glycine (G) hinge residues proximal to the PDZ binding motif, and positively charged residues (K, R, D, E) proximal to the hinge residues.

PDZ domains were originally identified as conserved sequence elements within the postsynaptic density protein PSD95/SAP90, the Drosophila tumor suppressor dlg-A, and the tight junction protein ZO-1. Although originally referred to as GLGF or DHR motifs, they are now known by an acronym representing these first three PDZ-containing proteins (PSD95/DLG/ZO-1). These 80-90 amino acid sequences have now been identified in well over 75 proteins and are characteristically expressed in multiple copies within a single protein. Thus, in some aspects, the provided polypeptide can inhibit the binding of an alpha Connexin to a protein comprising a PDZ domain. The PDZ domain is a specific type of protein-interaction module that has a structurally well-defined interaction ‘pocket’ that can be filled by a PDZ-binding motif, referred to herein as a “PDZ motif”. PDZ motifs are consensus sequences that are normally, but not always, located at the extreme intracellular carboxyl terminus. Four types of PDZ motifs have been classified: type I (S/T-x-Φ), type II (Φ-x-Φ), type III (Ψ-x-Φ) and type IV (D-x-V), where x is any amino acid, Φ is a hydrophobic residue (V, I, L, A, G, W, C, M, F) and Ψ is a basic, hydrophilic residue (H, R, K). (Songyang, Z., et al. 1997. Science 275, 73-77). Thus, in some aspects, the provided polypeptide comprises a type II PDZ binding motif.

It is noted that the 18 carboxy-terminal-most amino acid sequence of alpha Cx37 represents an exceptional variation on the ACT peptide theme. The Cx37 ACT-like sequence is GQKPPSRPSSSASKKQ*YV (SEQ ID NO: 43). Thus the carboxy terminal 4 amino acids of Cx37 conform only in part to a type II PDZ binding domain. Instead of a classical type II PDZ binding domain, Cx37 has a neutral Q* at position 2 where a hydrophobic amino acid would be expected. As such Cx37 comprises what might be termed a type II PDZ binding domain-like sequence. Nonetheless, Cx37 strictly maintains all other aspects of ACT peptide organization including clustered serine residues, frequent R and K residues and a P-rich sequence proximal to the PDZ binding domain-like sequence. Given this overall level of conservation of ACT-like organization in common with the other >70 alpha Connexins listed above, it is understood that the Cx37 ACT-like carboxy terminus functions in the provided capacity.

For comparison, the beta Connexin Cx26 is shown in Table 2. Cx26 has no carboxyl terminal type II PDZ binding motif, less than 30% of the carboxyl terminal most amino acids comprise S, T, R, D or E residues; it has no evidence of motifs proximal to a type II PDZ binding motif or PDZ binding like motif containing clusters of P and G hinge residues; and no evidence of clustered, repeat-like motifs of serine and threonine phosphor-amino acids. Cx26 does have three Lysine (K) residues, clustered one after the other near the carboxy terminus of the sequence. However, no alpha Connexin surveyed in the >70 alpha Connexins listed above was found to display this feature of three repeated K residues domain at carboxy terminus (Cx26 is a beta connexin, thus by definition does not have an ACT domain).

As provided herein, the unique functional characteristics of this relatively short stretch of amino acids encompass the disclosed roles in treating or preventing pathologies involving epithelial permeabilization and/or neovascularization. Thus, in some aspects, the provided polypeptide comprises a type II PDZ binding motif (Φ-x-Φ); wherein x=any amino acid and Φ=a Hydrophobic amino acid). In some aspects, greater than 50%, 60%, 70%, 80%, 90% of the amino acids of the provided ACT polypeptide is comprised one or more of Proline (P), Glycine (G), phospho-Serine (S), phospho-Threonine (T), Arginine (R), Lysine (K), Aspartic acid (D), or Glutamic acid (E) amino acid residues.

The amino acids Proline (P), Glycine (G), Arginine (R), Lysine (K), Aspartic acid (D), and Glutamic acid (E) are necessary determinants of protein structure and function. Proline and Glycine residues provide for tight turns in the 3D structure of proteins, enabling the generation of folded conformations of the polypeptide required for function. Charged amino acid sequences are often located at the surface of folded proteins and are necessary for chemical interactions mediated by the polypeptide including protein-protein interactions, protein-lipid interactions, enzyme-substrate interactions and protein-nucleic acid interactions. Thus, in some aspects Proline (P) and Glycine (G) Lysine (K), Aspartic acid (D), and Glutamic acid (E) rich regions proximal to the type II PDZ binding motif provide for properties necessary to the provided actions of ACT peptides. In some aspects, the provided polypeptide comprises Proline (P) and Glycine (G) Lysine (K), Aspartic acid (D), and/or Glutamic acid (E) rich regions proximal to the type II PDZ binding motif.

Phosphorylation is the most common post-translational modification of proteins and is crucial for modulating or modifying protein structure and function. Aspects of protein structure and function modified by phosphorylation include protein conformation, protein-protein interactions, protein-lipid interactions, protein-nucleic acid interactions, channel gating, protein trafficking and protein turnover. Thus, in some aspects the phospho-Serine (S) and/or phospho-Threonine (T) rich sequences are necessary for modifying the function of ACT peptides, increasing or decreasing efficacy of the polypeptides in their provided actions. In some aspects, the provided polypeptide comprise Serine (S) and/or phospho-Threonine (T) rich sequences or motifs.

In another example, a methionine occurs near the amino terminus of the ACT sequence of zebrafish Cx43 (Table 2). In addition to encoding methionine, the methionine base pair triplet is an alternate translation start site. If translation initiated from this methionine, the sequence SSRARPDDLDV (SEQ ID NO:90), would be produced. This translation product maintains all the conserved and distinctive features of a canonical ACT peptide. Specifically this peptide comprises a carboxy terminal type II PDZ binding domain and has a domain enriched in P, R and D residues proximal to the PDZ binding domain. In addition, the sequence comprises a clustered S motif, with potential to modulate ACT peptide function at its amino terminal. This raises the interesting prospect that animals with high tissue/organ regeneration potential such as fish may translate ACT peptides sequences directly.

Thus, in some aspects, the provided polypeptide comprises the c-terminal sequence of human Cx43. Thus, the provided polypeptide can comprise the amino acid sequence SEQ ID NO:1 or SEQ ID NO:2. The polypeptide can comprise 9 amino acids of the carboxy terminus of human Cx40. Thus, the polypeptide can comprise the amino acid sequence SEQ ID NO:5. In other aspects, the provided polypeptide does not comprises the c-terminal sequence of human Cx43. In some aspects, the provided polypeptide comprises or consists of the amino acid sequence SEQ ID NO:1 or SEQ ID NO:2.

When specific proteins are referred to herein, variants, derivatives, and fragments are contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known and include, for example, M13 primer mutagenesis and PCR mutagenesis. Amino acid substitutions are typically of single residues, but can occur at a number of different locations at once; insertions usually will be on the order of about from 1 to 10 amino acid residues. Deletions or insertions preferably are made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure unless such a change in secondary structure of the mRNA is desired. Substitutional variants are those in which at least one residue has been removed and a different residue inserted in its place. Such substitutions generally are made in accordance with the following Table 3 and are referred to as conservative substitutions.

TABLE 3
Amino Acid Substitutions

| Original Residue | Exemplary Substitutions |
|---|---|
| Ala | Ser |
| Arg | Lys |
| Asn | Gln |
| Asp | Glu |
| Cys | Ser |
| Gln | Asn |
| Glu | Asp |
| Gly | Pro |
| His | Gln |
| Ile | Leu; Val |
| Leu | Ile; Val |
| Lys | Arg; Gln |
| Met | Leu; Ile |
| Phe | Met; Leu; Tyr |
| Pro | Gly |
| Ser | Thr |
| Thr | Ser |
| Trp | Tyr |
| Tyr | Trp; Phe |
| Val | Ile; Leu |

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations shown in Table 3. Conservatively substituted variations of each explicitly disclosed sequence are included within the polypeptides provided herein.

Typically, conservative substitutions have little to no impact on the biological activity of a resulting polypeptide. In a particular example, a conservative substitution is an amino acid substitution in a peptide that does not substantially affect the biological function of the peptide. A peptide can include one or more amino acid substitutions, for example 2-10 conservative substitutions, 2-5 conservative substitutions, 4-9 conservative substitutions, such as 2, 5 or 10 conservative substitutions.

A polypeptide can be produced to contain one or more conservative substitutions by manipulating the nucleotide sequence that encodes that polypeptide using, for example, standard procedures such as site-directed mutagenesis or PCR. Alternatively, a polypeptide can be produced to contain one or more conservative substitutions by using standard peptide synthesis methods. An alanine scan can be used to identify which amino acid residues in a protein can tolerate an amino acid substitution. In one example, the biological activity of the protein is not decreased by more than 25%, for example not more than 20%, for example not more than 10%, when an alanine, or other conservative amino acid (such as those listed below), is substituted for one or more native amino acids.

Further information about conservative substitutions can be found in, among other locations, in Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988) and in standard textbooks of genetics and molecular biology.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that there are numerous amino acid and peptide analogs which can be incorporated into the disclosed compositions. For example, there are numerous D amino acids or amino acids which have a different functional substituent than the amino acids shown in Table 3. The opposite stereoisomers of naturally occurring peptides are disclosed, as well as the stereoisomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994), all of which are herein incorporated by reference at least for material related to amino acid analogs).

Molecules can be produced that resemble polypeptides, but which are not connected via a natural peptide linkage. For example, linkages for amino acids or amino acid analogs can include CH₂NH—, —CH₂S—, —CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. It is understood that peptide analogs can have more than one atom between the bond atoms, such as b-alanine, g-aminobutyric acid, and the like.

Amino acid analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, greater ability to cross biological barriers (e.g., gut, blood vessels, blood-brain-barrier), and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

Thus, the provided polypeptide can comprise a conservative variant of the c-terminus of an alpha Connexin (ACT). As shown in Table 4, an example of a single conservative substitution within the sequence SEQ ID NO:2 is given in the sequence SEQ ID NO:3. An example of three conservative substitutions within the sequence SEQ ID NO:2 is given in the sequence SEQ ID NO:4. Thus, the provided polypeptide can comprise the amino acid SEQ ID NO:3 or SEQ ID NO:4.

TABLE 4
ACT Polypeptide Variants

| Sequence | SEQ ID NO |
|---|---|
| RPRPDDLEI | SEQ ID NO: 2 |
| RPRPDDLEV | SEQ ID NO: 3 |
| RPRPDDVPV | SEQ ID NO: 4 |
| SSRASSRASSRPRPDDLEV | SEQ ID NO: 44 |
| RPKPDDLEI | SEQ ID NO: 45 |
| SSRASSRASSRPKPDDLEI | SEQ ID NO: 46 |
| RPKPDDLDI | SEQ ID NO: 47 |
| SSRASSRASSRPRPDDLDI | SEQ ID NO: 48 |
| SSRASTRASSRPRPDDLEI | SEQ ID NO: 49 |
| RPRPEDLEI | SEQ ID NO: 50 |
| SSRASSRASSRPRPEDLEI | SEQ ID NO: 51 |
| GDGKNSVWV | SEQ ID NO: 52 |
| SKAGSNKSTASSKSGDGKNSVWV | SEQ ID NO: 53 |
| GQKPPSRPSSSASKKLYV | SEQ ID NO: 54 |

It is understood that one way to define any variants, modifications, or derivatives of the disclosed genes and proteins herein is through defining the variants, modification, and derivatives in terms of sequence identity (also referred to herein as homology) to specific known sequences. Specifically disclosed are variants of the nucleic acids and polypeptides herein disclosed which have at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the stated or known sequence. Those of skill in the art readily understand how to determine the sequence identity of two proteins or nucleic acids. For example, the sequence identity can be calculated after aligning the two sequences so that the sequence identity is at its highest level.

Another way of calculating sequence identity can be performed by published algorithms. Optimal alignment of sequences for comparison may be conducted by the local sequence identity algorithm of Smith and Waterman Adv. Appl. Math. 2: 482 (1981), by the sequence identity alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection. These references are incorporated herein by reference in their entirety for the methods of calculating sequence identity.

The same types of sequence identity can be obtained for nucleic acids by, for example, the algorithms disclosed in Zuker, M. Science 244:48-52, 1989, Jaeger et al. Proc. Nat. Acad. Sci. USA 86:7706-7710, 1989, Jaeger et al. Methods Enzymol. 183:281-306, 1989 which are herein incorporated by reference for at least material related to nucleic acid alignment.

Thus, the provided polypeptide can comprise an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the c-terminus of an alpha Connexin (ACT). Thus, in some aspects, the provided polypeptide comprises an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to SEQ ID NO:1, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:90, SEQ ID NO:91, or SEQ ID NO:92. As an example, provided is a polypeptide (SEQ ID NO:4) having 66% sequence identity to the same stretch of 9 amino acids occurring on the carboxy-terminus of human Cx43 (SEQ ID NO:2).

The herein provided polypeptides can be added directly to a tissue in a subject. However, efficiency of cytoplasmic localization of the provided polypeptide is enhanced by cellular internalization transporter chemically linked in cis or trans with the polypeptide. Efficiency of cell internalization transporters are enhanced further by light or co-transduction of cells with Tat-HA peptide.

Thus, the provided polypeptide can comprise a cellular internalization transporter or sequence. The cellular internalization sequence can be any internalization sequence known or newly discovered in the art, or conservative variants thereof. Non-limiting examples of cellular internalization transporters and sequences include Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol) (see Table 5).

TABLE 5
Cell Internalization Transporters

| Name | Sequence | SEQ ID NO |
|---|---|---|
| Antp | RQPKIWFPNRRKPWKK | (SEQ ID NO: 7) |
| HIV-Tat | GRKKRRQRPPQ | (SEQ ID NO: 14) |
| Penetratin | RQIKIWFQNRRMKWKK | (SEQ ID NO: 15) |
| Antp-3A | RQIAIWFQNRRMKWAA | (SEQ ID NO: 16) |
| Tat | RKKRRQRRR | (SEQ ID NO: 17) |
| Buforin II | TRSSRAGLQFPVGRVHRLLRK | (SEQ ID NO: 18) |
| Transportan | GWTLNSAGYLLGKINKALAALAKKIL | (SEQ ID NO: 19) |
| model amphipathic | KLALKLALKALKAALKLA | (SEQ ID NO: 20) |
| peptide (MAP) | | |
| K-FGF | AAVALLPAVLLALLAP | (SEQ ID NO: 21) |
| Ku70 | VPMLK-PMLKE | (SEQ ID NO: 22) |
| Prion | MANLGYWLLALFVTMWTDVGLCKKRPKP | (SEQ ID NO: 23) |
| pVEC | LLIILRRRIRKQAHAHSK | (SEQ ID NO: 24) |
| Pep-1 | KETWWETWWTEWSQPKKKRKV | (SEQ ID NO: 25) |
| SynB1 | RGGRLSYSRRRFSTSTGR | (SEQ ID NO: 26) |
| Pep-7 | SDLWEMMMVSLACQY | (SEQ ID NO: 27) |
| HN-1 | TSPLNIHNGQKL | (SEQ ID NO: 28) |
| BGSC (Bis- Guanidiniurn- Sperrnidine- Cholesterol) | 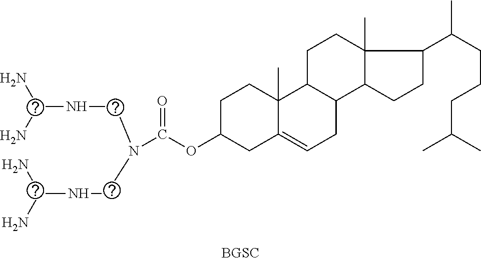 [SMILES: CC(C)CCCC(C)C1CCC2C3CC=C4CC(OC(=O)N(CNC(N)N)CNC(N)N)CCC4(C)C3CCC12C] | |
| BGTC (Bis- Guanidinium-Tren- Cholesterol) | 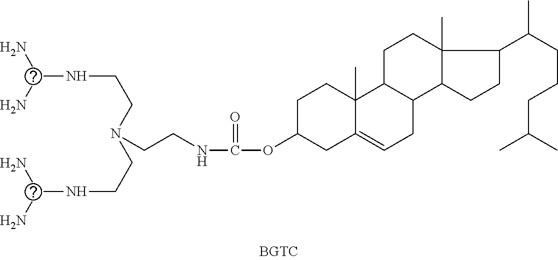 [SMILES: CC(C)CCCC(C)C1CCC2C3CC=C4CC(OC(=O)NCCN(CCNC(N)N)CCNC(N)N)CCC4(C)C3CCC12C] | |

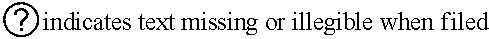

Thus, the provided polypeptide can further comprise the amino acid sequence SEQ ID NO:7, SEQ ID NO:14 (Bocci, M. et al. 2000. Nat. Med. 6, 1362-1367), SEQ ID NO:15 (Derossi, D., et al. 1994. Biol. Chem. 269, 10444-10450), SEQ ID NO:16 (Fischer, P. M. et al 2000. J. Pept. Res. 55, 163-172), SEQ ID NO:17 (Frankel, A. D. & Pabo, C. O. 1988. Cell 55, 1189-1193; Green, M. & Loewenstein, P. M. 1988. Cell 55, 1179-1188), SEQ ID NO:18 (Park, C. B., et al. 2000. Proc. Natl Acad. Sci. USA 97, 8245-8250), SEQ ID NO:19 (Pooga, M., et al. 1998. FASEB J. 12, 67-77), SEQ ID NO:20 (Oehlke, J. et al. 1998. Biochim. Biophys. Acta. 1414, 127-139), SEQ ID NO:21 (Lin, Y. Z., et al. 1995. J. Biol. Chem. 270, 14255-14258), SEQ ID NO:22 (Sawada, M., et al. 2003. Nature Cell Biol. 5, 352-357), SEQ ID NO:23 (Lundberg, P. et al. 2002. Biochem. Biophys. Res. Commun. 299, 85-90), SEQ ID NO:24 (Elmquist, A., et al. 2001. Exp. Cell Res. 269, 237-244), SEQ ID NO:25 (Morris, M. C., et al. 2001. Nature Biotechnol. 19, 1173-1176), SEQ ID NO:26 (Rousselle, C. et al. 2000. Mol. Pharmacol. 57, 679-686), SEQ ID NO:27 (Gao, C. et al. 2002. Bioorg. Med. Chem. 10, 4057-4065), or SEQ ID NO:28 (Hong, F. D. & Clayman, G. L. 2000. Cancer Res. 60, 6551-6556). The provided polypeptide can further comprise BGSC (Bis-Guanidinium-Spermidine-Cholesterol) or BGTC (Bis-Guanidinium-Tren-Cholesterol) (Vigneron, J. P. et al. 1998. Proc. Nat. Acad. Sci. USA. 93, 9682-9686). The preceding references are hereby incorporated herein by reference in their entirety for the teachings of cellular internalization vectors and sequences. Any other internalization sequences now known or later identified can be combined with a peptide of the invention.

The provided polypeptide can comprise any ACT sequence (e.g, any of the ACT peptides disclosed herein) in combination with any of the herein provided cell internalization sequences. Examples of said combinations are given in Table 6. Thus, the provided polypeptide can comprise an Antennapedia sequence comprising amino acid sequence SEQ ID NO:7. Thus, the provided polypeptide can comprise the amino acid sequence SEQ ID NO:8, SEQ ID NO:9, SEQ ID NO:10, SEQ ID NO:11, or SEQ ID NO:12.

TABLE 6
ACT Polypeptides with Cell Internalization Sequences (CIS)

| CIS/ | | |
|---|---|---|
| ACT | Sequence | SEQ ID NO |
| Antp/ | RQPKIWFPNRRKPWKK PSSRASSRASSRPRPDDLEI | SEQ ID NO: 8 |
| ACT 2 | | |
| Antp/ | RQPKIWFPNRRKPWKK RPRPDDLEI | SEQ ID NO: 9 |
| ACT 1 | | |
| Antp/ | RQPKIWFPNRRKPWKK RPRPDDLEV | SEQ ID NO: 10 |
| ACT 3 | | |
| Antp/ | RQPKIWFPNRRKPWKK RPRPDDVPV | SEQ ID NO: 11 |
| ACT 4 | | |
| Antp/ | RQPKIWFPNRRKPWKK KARSDDLSV | SEQ ID NO: 12 |
| ACT 5 | | |
| HIV-Tat/ | GRKKRRQRPPQ RPRPDDLEI | SEQ ID NO: 56 |
| ACT 1 | | |
| Penetratin/ | RQIKIWFQNRRMKWKK RPRPDDLEI | SEQ ID NO: 57 |
| ACT 1 | | |
| Antp-3A/ | RQIAIWFQNRRMKWAA RPRPDDLEI | SEQ ID NO: 58 |
| ACT 1 | | |
| Tat/ | RKKRRQRRR RPRPDDLEI | SEQ ID NO: 59 |
| ACT 1 | | |
| Buforin II/ | TRSSRAGLQFPVGRVHRLLRK RPRPDDLEI | SEQ ID NO: 60 |
| ACT 1 | | |
| Transportan/ | GWTLNSAGYLLGKINKALAALAKKIL RPRPDDLEI | SEQ ID NO: 61 |
| ACT 1 | | |
| MAP/ | KLALKLALKALKAALKLA RPRPDDLEI | SEQ ID NO: 62 |
| ACT 1 | | |
| K-FGF/ | AAVALLPAVLLALLAP RPRPDDLEI | SEQ ID NO: 63 |
| ACT 1 | | |
| Ku70/ | VPMLKPMLKE RPRPDDLEI | SEQ ID NO: 64 |
| ACT 1 | | |
| Prion/ | MANLGYWLLALFVTMWTDVGLCKKRPKP | SEQ ID NO: 65 |
| ACT 1 | RPRPDDLEI | |
| pVEC/ | LLIILRRRIRKQAHAHSK RPRPDDLEI | SEQ ID NO: 66 |
| ACT 1 | | |
| Pep-1/ | KETWWETWWTEWSQPKKKRKV RPRPDDLEI | SEQ ID NO: 67 |
| ACT 1 | | |
| SynB1/ | RGGRLSYSRRRFSTSTGR RPRPDDLEI | SEQ ID NO: 68 |
| ACT 1 | | |
| Pep-7/ | SDLWEMMMVSLACQY RPRPDDLEI | SEQ ID NO: 69 |
| ACT 1 | | |
| HN-1/ | TSPLNIHNGQKL RPRPDDLEI | SEQ ID NO: 70 |
| ACT 1 | | |

Also provided are isolated nucleic acids encoding the polypeptides provided herein. The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (e.g., a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, e.g., other types of RNA molecules or polypeptide molecules.

Thus, provided is an isolated nucleic acid encoding a polypeptide comprising the amino acid sequence SEQ ID NO:1, SEQ ID NO:2, SEQ ID NO:3, SEQ ID NO:4, SEQ ID NO:5, SEQ ID NO:7, SEQ ID NO:8, SEQ ID NO:9, SEQ ID NO:10, SEQ ID NO:11, or SEQ ID NO:12.

Thus, the provided nucleic acid can comprise the nucleic acid sequence SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, or SEQ ID NO:89.

Provided is a composition comprising one or more of the herein provided polypeptides, nucleic acids, or vectors in a pharmaceutically acceptable carrier. Thus, provided is a composition comprising a combination of two or more of any of the herein provided ACT polypeptides in a pharmaceutically acceptable carrier. For example, provided is a composition comprising SEQ ID NO:1 and SEQ ID NO:5 in a pharmaceutically acceptable carrier.

By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Organ or tissue transplantation is the moving of an organ or tissue from one body to another or from a donor site to another location on the person's own body, to replace the recipient's damaged or absent organ or tissue.

Organs and/or tissues that are transplanted within the same person's body are called autografts. Sometimes an autograft is done to remove the tissue and then treat it or the person before returning it (examples include stem cell autograft and storing blood in advance of surgery).

Organs and/or tissues that are transplanted between two genetically non-identical members of the same species are called allografts. Allografts can either be from a living or cadaveric source. Most human tissue and organ transplants are allografts. Due to the genetic difference between the organ and the recipient, the recipient's immune system will identify the organ as foreign and attempt to destroy it, causing transplant rejection.

Isografts are a subset of allografts in which organs or tissues are transplanted from a donor to a genetically identical recipient (such as an identical twin). Isografts are differentiated from other types of transplants because while they are anatomically identical to allografts, they do not trigger an immune response.

A transplant of organs or tissue from one species to another is called a xenograft. An example is porcine heart valve transplant, which is quite common and successful. Another example is attempted piscine-primate (fish to non-human primate) transplant of islet (i.e. pancreatic or insular) tissue. Xenotransplantion is often an extremely dangerous type of transplant because of the increased risk of non-compatibility, rejection, and disease carried in the tissue.

Organs that can be transplanted include the heart, kidneys, liver, lungs, pancreas, intestine, and thymus. Tissues include bones, tendons (both referred to as musculoskeletal grafts), cornea, skin, heart valves, nerves and veins. Worldwide, the kidneys are the most commonly transplanted organs, followed by the liver and then the heart. Cornea and musculoskeletal grafts are the most commonly transplanted tissues; these outnumber organ transplants by more than tenfold.

Organ donors may be living, brain dead, or dead via circulatory death. Tissue may be recovered from donors who die of circulatory death, as well as of brain death—up to 24 hours past the cessation of heartbeat. Unlike organs, most tissues (with the exception of corneas) can be preserved and stored for up to five years, meaning they can be “banked”.

The polypeptides may be contacted with the organs or tissues designated for transplant by any means known in the art. For example, in some embodiments, the polypeptides are added to a cold storage solution and the tissue or organ is incubated in the solution comprising the peptide. In some embodiments, the polypeptides are added to a solution that is used to perfuse an organ or tissue prior to transplant. In some embodiments, the polypeptide is added to both the storage and the perfusion solution. Storage and/or perfusion solutions are known in the art and include, without limitation, University of Wisconsin (UW) solution, Histidine-tryptophan-ketoglutarate (HTK) solution, Collins solution, Belzer solution, Euro-Collins solution, Celsior solution, Kyoto solution, Institut Georges Lopez (IGL-1) solution, Marxhall's hypertonic citrate (HOC) solution, sucrose phosphate buffer, and Bretschneider's solution; and any modification thereof.

The concentration of the polypeptide added to the solution may range from about 0.01 μM to about 100 μM. For example, the concentration of the polypeptide in the solution may be about 0.01 μM, about 0.05 μM, about 0.1 μM, about 0.15 μM, about 0.2 μM, about 0.3 μM, about 0.4 μM, about 0.5 μM, about 0.6 μM, about 0.7 μM, about 0.8 μM, about 0.9 μM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 50 μM, about 75 μM, or about 100 μM. In some embodiments, the polypeptide is added to the solution in a concentration of from about 0.1 μL/mL to about 1000 μL/mL. For example, in some embodiments, concentration is about 0.1 μL/mL, about 1 μL/mL, about 5 μL/mL, about 10 μL/mL, about 100 μL/mL, about 200 μL/mL, about 300 μL/mL, about 400 μL/mL, about 500 μL/mL, about 750 μL/mL, or about 1000 μL/mL. In some embodiments, the concentration added to the solution is a concentration that results in an effective amount of peptide present in the organ that ranges from about 0.1 μM to about 100 μM, for example.

In some embodiments, the compositions provided herein are organ preservation solutions comprising a polypeptide comprising the carboxy-terminal amino acid sequence of an alpha connexin, or a conservative variant thereof (e.g., a polypeptide having an amino acid sequence according to SEQ ID NO:1, 2, 3, 4, or 5). For example, in some embodiments, the compositions provided herein comprise UW solution and a polypeptide having an amino acid sequence according to SEQ ID NO: 2. For example, in some embodiments, the compositions provided herein comprise UW solution and a polypeptide having an amino acid sequence according to SEQ ID NO: 9 (ACT1). In some embodiments, the solution comprises, for example, a polypeptide comprising the carboxy-terminal amino acid sequence of an alpha connexin, or a conservative variant thereof (e.g. SEQ ID NO: 1, 2, 3, 4, or 5), potassium, sodium, magnesium, lactobionate, phosphate, sulphate, raffinose, adenosine, allopurinol, glutathione, insulin, dexamethasone, hydroxyethyl starch (HES), and/or Bactrim. In further embodiments, UW solution comprises about 135 mmol/L potassium, about 35 mmol/L sodium, about 5 mmol/L magnesium, about 100 mmol/L lactobionate, about 25 mmol/L phosphate, about 5 mmol/L sulphate, about 30 mmol/L raffinose, about 5 mmol/L adenosine, about 1 mmol/L allopurinol, about 3 mmol/L glutathione, about 100 U/L insulin, about 8 mg/L dexamethasone, about 50 g/L HES, and/or about 0.5 ml/L Bactrim. The skilled artisan will recognize that any organ or tissue preservation solution known in the art can be used in the methods and compositions disclosed herein. Accordingly, the alpha connexin polypeptides provided herein can be added to any organ or tissue preservation solution or perfusion solution known in the art in order to improve the preservation properties of the organ or tissue preservation solution. In some embodiments, the alpha connexin polypeptides provided herein unexpectedly are capable of rescuing organs or tissues that would otherwise not be suitable for transplant. For example, some organs or tissues become damaged and/or lose functionality during the organ harvesting and cold storage process, such that they cannot be used due to the high degree of risk that the organ will fail and/or be rejected soon after transplant. In some embodiments, the present disclosure provides the surprisingly efficient and improved method for preserving organs or tissues to increase the number of organs and tissues that can be transplanted rather than going to waste; and/or the surprisingly efficient and improved method for rescuing organs or tissues that have lost some functionality and/or have become damaged during the organ harvesting and/or cold storage period. In some embodiments, the present disclosure provides compositions and methods for preserving organs and tissues for transplantation that are effective using surprisingly low amounts and concentrations of an alpha connexin peptide, e.g., ACT1.

In some embodiments, the present disclosure provides compositions comprising one or more organ (e.g. heart, kidneys, liver, lungs, pancreas, intestine, and thymus) or tissue for transplantation into a recipient, and a polypeptide as provided herein (e.g., a polypeptide comprising the carboxy-terminus of an alpha connexin, or a conservative variant thereof). In further embodiments, the composition comprises one or more organ or tissue, an alpha connexin polypeptide as provided herein, and an organ preservation solution.

In one aspect, the present disclosure provides compositions and methods for preserving an organ for organ transplantation comprising incubating the organ with a solution comprising a polypeptide comprising the carboxy-terminal amino acid sequence of an alpha connexin, or a conservative variant thereof, as provided herein. In some embodiments, the present disclosure provides compositions and methods for preserving an organ for organ transplantation comprising perfusing the organ with a solution comprising a polypeptide comprising the carboxy-terminal amino acid sequence of an alpha connexin, or a conservative variant thereof, as provided herein. In some embodiments, the present disclosure provides compositions and methods for preserving a tissue for tissue transplantation, comprising incubating the tissue with a solution comprising a polypeptide comprising the carboxy-terminal amino acid sequence of an alpha connexin, or a conservative variant thereof, as provided herein. In some embodiments, the organ is lung tissue, and the lung is contacted with the alpha connexin peptide via nebulization of the donor.

In some embodiments, the methods and solutions provided herein inhibit endothelial and/or epithelial cellular injury. In some embodiments, the methods and solutions provided herein inhibit mitochondrial oxidant production. In some embodiments, the polypeptide inhibits inflammation. In some embodiments, the polypeptide inhibits pro-inflammatory cytokine release. Pro-inflammatory cytokines are known in the art and include, for example, IL-8, IFNγ, TNF, IL-12, IL-6, IL-1β, IL-2, and IL-17.

In some embodiments, the methods and compositions provided herein rescue marginal organs and tissues for transplantation. The term “marginal,” as used herein to describe an organ or tissue, is used interchangeably with “sub-optimal” and the like, and refers to organs and tissues that would otherwise not be suitable for transplantation or would otherwise fail or become rejected shortly after transplantation due to, for example, long storage time and/or damage to the organ or tissue prior to, during, or after organ harvest. For example, marginal organs or tissues that may have failed functionally and/or been rejected shortly after transplant; or marginal organs or tissues that may have been deemed unsuitable for transplantation due to cellular injury, can be rescued by contacting the organ or tissue with the solution or composition provided herein, such that cellular injury is minimized or reversed and the organ or tissue transplant can proceed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

As used herein and in the appended claims, the singular forms “a,” “an,” and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

As used herein, “inhibit,” “inhibiting,” and “inhibition” mean to decrease an activity, response, condition, disease, or other biological parameter. This can include, but is not limited to, the complete loss of activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, “subject” includes, but is not limited to, animals, plants, bacteria, viruses, parasites and any other organism or entity that has nucleic acid. The subject may be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

The term “peptide” is used interchangeably herein with “polypeptide” and encompasses peptides comprising or consisting of an alpha connexin peptide described herein, and peptides comprising or consisting of an alpha connexin peptides that is linked to a cell penetration peptide. The term “cell penetration peptide” and the like is used interchangeably herein with the term “cellular internalization sequence” and the like. Thus, the peptides and polypeptides provided herein include ACT1 peptide, which is SEQ ID NO: 9 herein, and which comprises SEQ ID NO: 2 and an antennapedia sequence.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure.

### Example 1. ACT1 Pretreatment of Endothelial Cells Prevents Cold Storage and Reperfusion Injury while Limiting Pro-Inflammatory Cytokine Responses

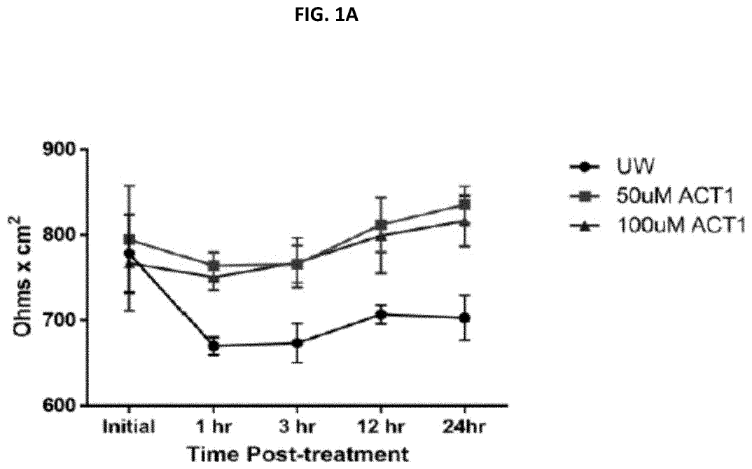

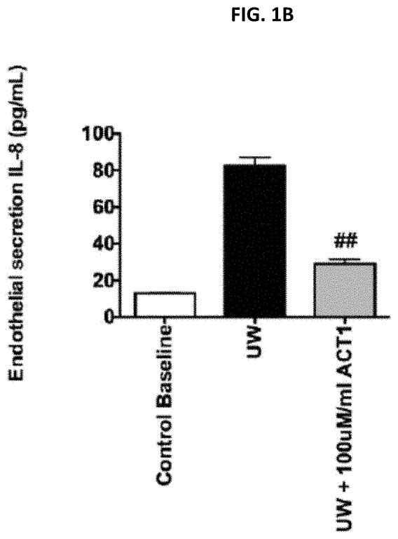

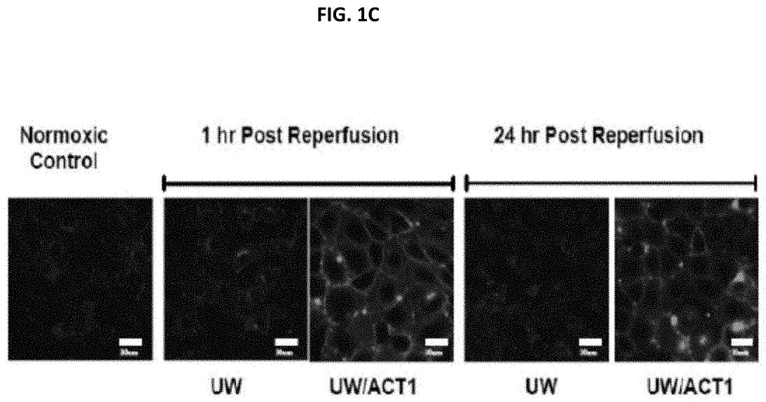

Endothelial cells (EC) were pre-treated with University of Wisconsin (UW) solution only, or UW solution comprising 50 μM or 100 μM ACT1. Cold storage and reperfusion injury, pro-inflammatory cytokine release, and gap junction stabilization (Cx43 expression) were measured. FIG. 1A shows that ACT1 pretreatment prevented cold storage and reperfusion injury at both concentrations (50 μM or 100 μM ACT1) at 1 hour, 3, hours, 12 hours, and 24 hours post-treatment, as measured by electrical resistance. FIG. 1B shows that ACT1 pretreatment significantly reduced IL-8 release from the ECs. FIG. 1C shows that pretreatment increased Cx43 expression at 1 hour and 24 hours post reperfusion.

### Example 2. ACT1 Augmentation of UW Solution Reduces Ischemic Reperfusion Injury (IRI)-Induced Heart Graft Injury and Early Post-Transplantation Immune Cell Infiltration

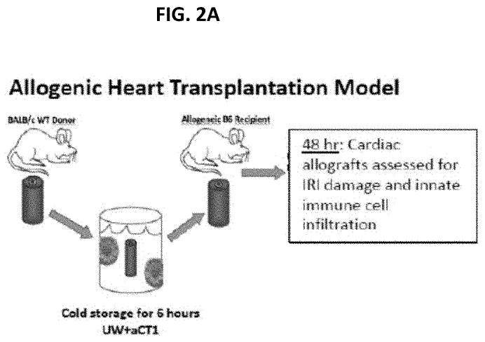

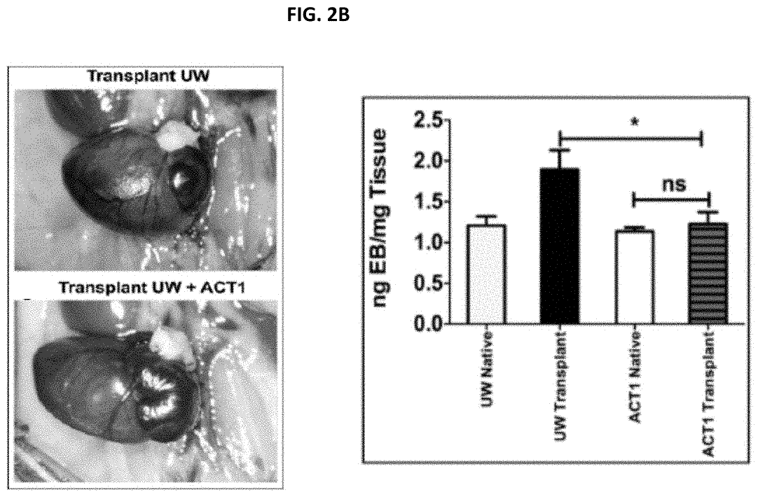

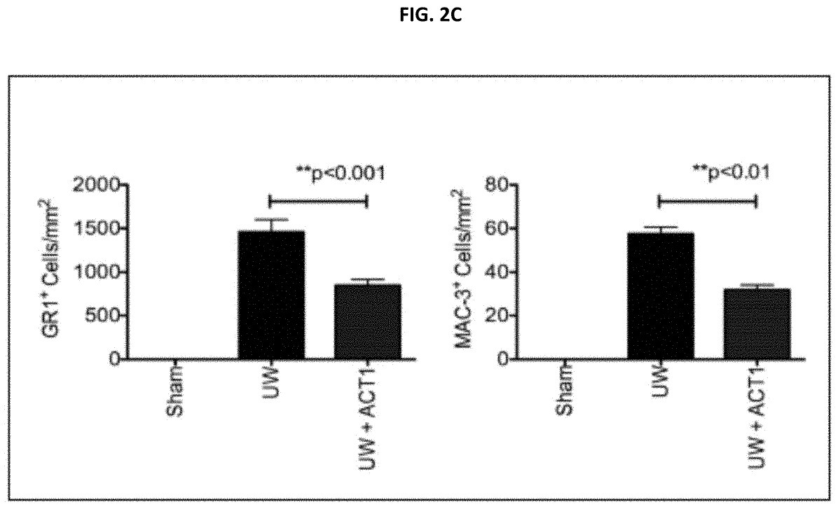

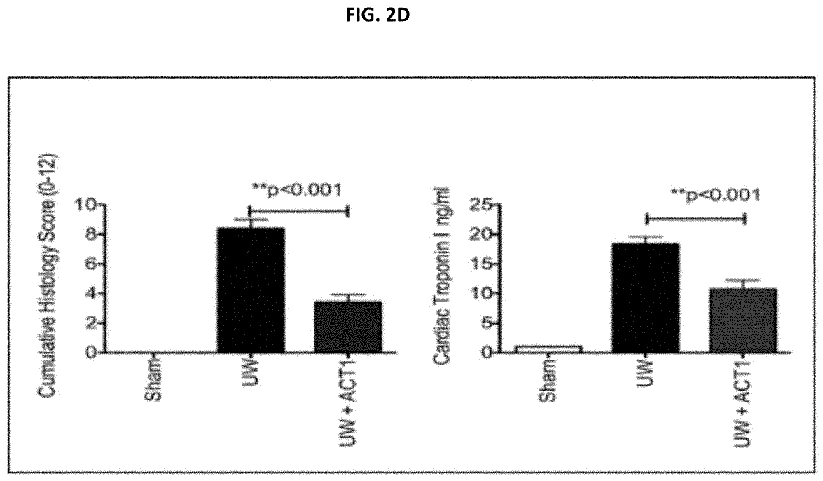

Heart allograft transplants were performed between Balb/c donors to B6 recipients as shown in FIG. 2A. Balb/c donor hearts were removed, perfused with UW solution and then static cold stored in either UW solution alone or UW solution supplemented with ACT1 peptide for 6 h at 4° C. Following storage, hearts were implanted into B6 recipients. To assess the impact of ACT1 peptide augmented cold storage on heart vascular permeability/damage, recipients were injected with Evan's Blue Dye immediately following reperfusion. Hearts were then harvested and assayed for Evan's Blue uptake.

As shown in FIG. 2B, the presence of ACT1 peptide in the cold storage solution significantly reduced the amount of Evan's Blue per mg tissue. Thus, ACT1 peptide reduced endothelial permeability post-transplantation. As shown in FIG. 2C, the presence of ACT1 peptide in the cold storage solution significantly reduced the number of neutrophils (as measured by GR1+ cells) and macrophages (as measured by MAC-3+ cells). Thus, ACT1 peptide reduced inflammation in the organ post-transplantation. As shown in FIG. 2D, the presence of ACT1 peptide in the cold storage solution significantly reduced IRI as measured by cumulative histology score and cardiac troponin I. Taken together, the results of the study showed that ACT1 peptide supplementation of UW solution minimizes cellular injury and inflammation, and improves overall donor organ quality

### Example 3. Donor Aorta ACT Pre-Treatment Ameliorates Neointimal Hyperplasia

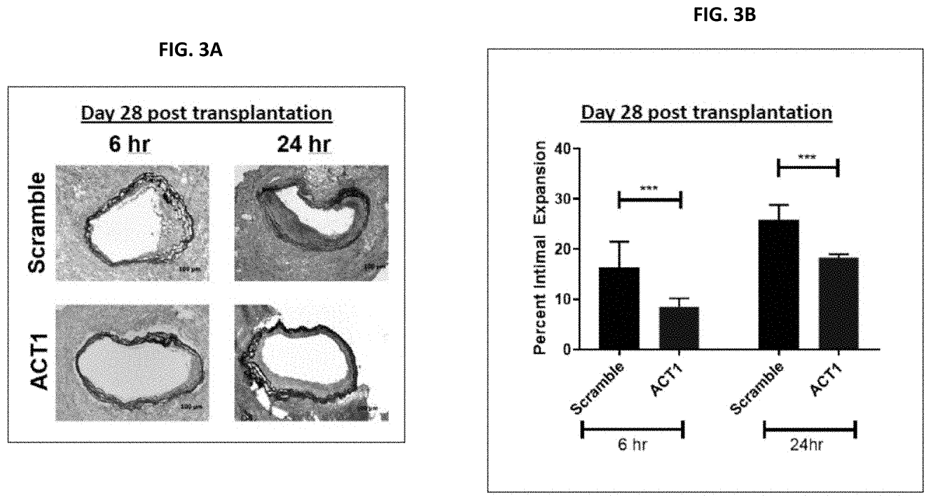

Heart allograft transplants were performed between Balb/c donors to B6 recipients as described above and shown in FIG. 2A. Aortic allografts were stored for 6 hours or 24 hours, and assessed on Day 28 after transplantation for neointimal hyperplasia (a hallmark of chronic rejection) and T cell infiltration. FIG. 3A shows that donor aorta ACT preservation solution treatment ameliorates neointimal hyperplasia following aorta transplantation. FIG. 3B shows that the percent intimal expansion is significantly reduced at 28 days post-treatment with ACT1 pre-treatment, including aortas stored for 24 hours.

### Example 4. ACT Efficacy in Pig Kidney Ex Vivo Perfusion Model

Pig kidneys were harvested and flushed with cold HTK solution, and then underwent 18 hours of cold static storage in HTK solution. At 18 hours, the kidneys were placed on a perfusion pump and perfused in cold HTK solution with or without 1 μM ACT1 (500 μL/mL). The control kidney in each pair was perfused with cold HTK and its pair was perfused with cold HTK supplemented with ACT1. While on pump, the resistance values were recorded and samples were collected of the perfusate solution every 30 minutes. At the end of 6 hours on the pump, kidneys were taken off of the pump and biopsies were collected and assessed for viability, function, and characteristics as described below.

Tubular necrosis (in this study, a measure of cell death) was assessed by basic histology/pathology methods and subjective scoring. Overall epithelial cell death was determined using EpCam as the marker. The relative expression of Vcam and ICam were determined via RT-qPCR in biopsied tissue at the end of each perfusion pump run. LDH (lactose dehydrogenase) was measured as a marker of tissue injury and was quantified using a colorimetric assay.

The goals of this study of ACT1 supplementation to perfusion solution included (1) increasing the donor pool, that is, maintain kidney functionality during cold storage to enable transplantation of kidneys deemed “marginal”; and (2) increasing lifetime of donor kidneys, that is, limit pre-graft tissue damage that decreases kidney function and exacerbates graft rejections.

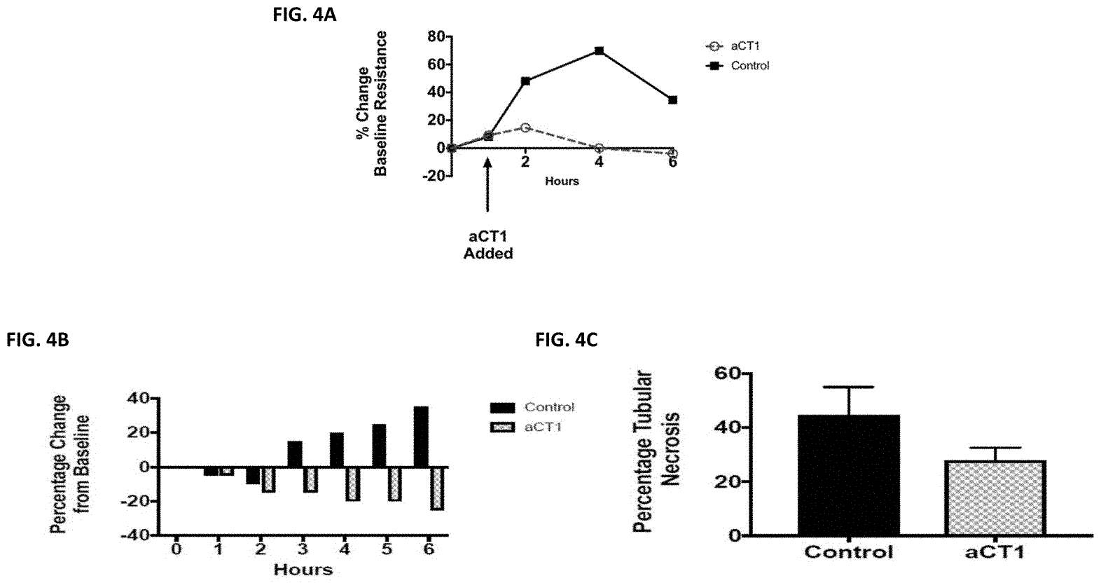

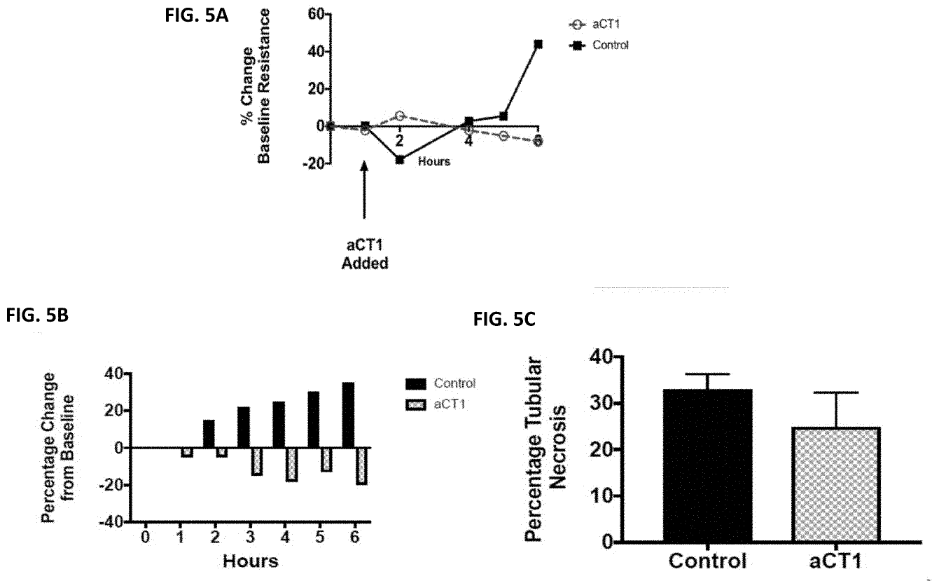

FIGS. 4A and 4B (match kidney donor pair #1) and 5A and 5 B (matched kidney donor pair #2) show that ACT1 prevents change in vascular renal resistance. FIG. 4C (matched kidney donor pair #1) and 5C (matched kidney donor pair #2) shows that ACT1 reduces tubular necrosis.

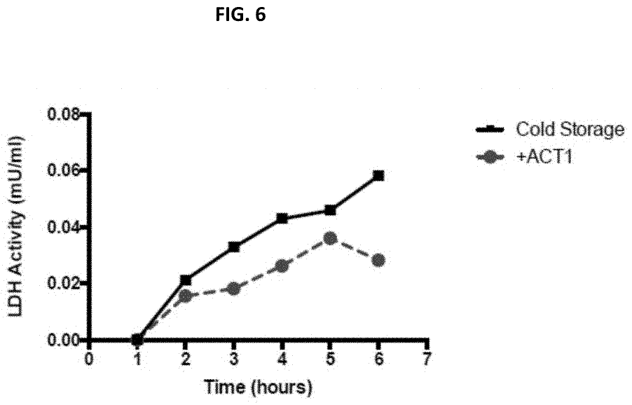

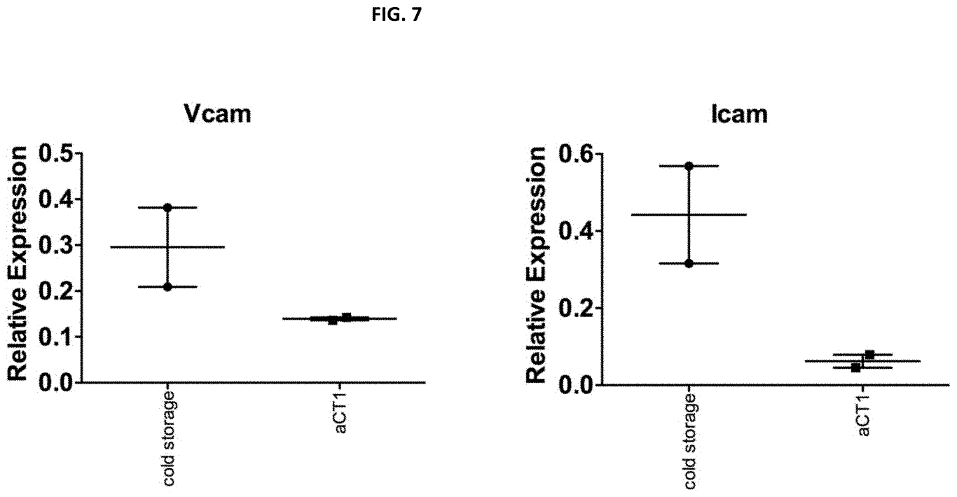

FIG. 6 shows that ACT1 decreases LDH activity in this model. FIG. 7 shows that ex vivo perfusion with ACT1-augmented HTK solution decreases endothelial cell activation as measured by Vcam or Icam expression.

Taken together, the results of the study showed that ex-vivo perfusion with ACT1-augmented solution decreases donor kidney injury. Surprisingly, these marginal kidneys were rescued by the addition of ACT1 peptide to the storage solution. Thus, ACT1 peptide not only improves function of kidneys and other organs that are transplanted, but is also useful for rescuing kidneys that would otherwise not be suitable for transplantation.

Sequences

SEQ ID NO: 1 (ACT 2)
PSSRASSRASSRPRPDDLEI
SEQ ID NO: 2 (Cx43 portion of ACT1)
RPRPDDLEI
SEQ ID NO: 3 (ACT 3)
RPRPDDLEV
SEQ ID NO: 4 (ACT 4)
RPRPDDVPV
SEQ ID NO: 5 (ACT 5)
KARSDDLSV
SEQ ID NO: 6
aga cct cgg cct gat gac ctg gag att
SEQ ID NO: 7 (Antp)
RQPKIWFPNRRKPWKK
SEQ ID NO: 8 (Antp/ACT 2)
RQPKIWFPNRRKPWKKPSSRASSRASSRPRPDDLEI
SEQ ID NO: 9 (ACT1)
RQPKIWFPNRRKPWKKRPRPDDLEI
SEQ ID NO: 10 (Antp/ACT 3)
RQPKIWFPNRRKPWKKRPRPDDLEV
SEQ ID NO: 11 (Antp/ACT 4)
RQPKIWFPNRRKPWKKRPRPDDVPV
SEQ ID NO: 12 (Antp/ACT 5)
RQPKIWFPNRRKPWKKKARSDDLSV
SEQ ID NO: 13 (encodes polypeptide of SEQ ID NO 9)
cgg cag ccc aag atc tgg ttc ccc aac cgg aag ccc
tgg aag cgg ccc ggc ccg acg acc tgg aga tc
SEQ ID NO: 14 (HIV-Tat)
GRKKRRQRPPQ
SEQ ID NO: 15 (Penetratin)
RQIKIWFQNRRMKWKK
SEQ ID NO: 16 (Antp-3A)
RQIAIWFQNRRMKWAA
SEQ ID NO: 17 (Tat)
RKKRRQRRR
SEQ ID NO: 18 (Buforin II)
TRSSRAGLQFPVGRVHRLLRK
SEQ ID NO: 19 (Transportan)
GWTLNSAGYLLGKINKALAALAKKIL
SEQ ID NO: 20 (model amphipathic peptide)
KLALKLALKALKAALKLA
SEQ ID NO: 21 (K-FGF)
AAVALLPAVLLALLAP
SEQ ID NO: 22 (Ku70)
VPMLK-PMLKE
SEQ ID NO: 23 (Prion)
MANLGYWLLALFVTMWTDVGLCKKRPKP
SEQ ID NO: 24 (pVEC)
LLIILRRRIRKQAHAHSK
SEQ ID NO: 25 (Pep-1)
KETWWETWWTEWSQPKKKRKV
SEQ ID NO: 26 (SynB1)
RGGRLSYSRRRFSTSTGR
SEQ ID NO: 27 (Pep-7)
SDLWEMMMVSLACQY
SEQ ID NO: 28 (HN-1)
TSPLNIHNGQKL
SEQ ID NO: 29 (Chick alpha Cx43 ACT)
PSRASSRASSRPRPDDLEI
SEQ ID NO: 30 (Human alpha Cx45)
GSNKSTASSKSPDPKNSVWI
SEQ ID NO: 31 (Chick alpha Cx45)
GSNKSSASSKSGDGKNSVWI
SEQ ID: 32 (Human alpha Cx46)
GRASKASRASSGRARPEDLAI
SEQ ID: 33 (Human alpha Cx46.6)
GSASSRDGKTVWI
SEQ ID NO: 34 (Chimp alpha Cx36)
PRVSVPNFGRTQSSDSAYV
SEQ ID NO: 35 (Chick alpha Cx36)
PRMSMPNFGRTQSSDSAYV
SEQ ID NO: 36 (Human alpha Cx47)
PRAGSEKGSASSRDGKTTVWI
SEQ ID NO: 37 (Human alpha Cx40)
GYHSDKRRLSKASSKARSDDLSV
SEQ ID NO: 38 (Human alpha Cx50)
PLSRLSKASSRARSDDLTV
SEQ ID NO: 39 (Human alpha Cx59)
PNHVVSLTNNLIGRRVPTDLQI
SEQ ID NO: 40 (Rat alpha Cx33)
PSCVSSSAVLTTICSSDQVVPVGLSSFYM
SEQ ID NO: 41 (Sheep alpha Cx44)
GRSSKASKSSGGRARAADLAI
SEQ ID NO: 42 (Human beta Cx26)
LCYLLIRYCSGKSKKPV
SEQ ID: 43 (Human alpha Cx37)
GQKPPSRPSSSASKKQ*YV
SEQ ID 44: (conservative Cx43 variant)
SSRASSRASSRPRPDDLEV
SEQ ID 45: (conservative Cx43 variant)
RPKPDDLEI,
SEQ ID 46: (conservative Cx43 variant)
SSRASSRASSRPKPDDLEI,
SEQ ID 47: (conservative Cx43 variant)
RPKPDDLDI
SEQ ID 48: (conservative Cx43 variant)
SSRASSRASSRPRPDDLDI
SEQ ID 49: (conservative Cx43 variant)
SSRASTRASSRPRPDDLEI
SEQ ID 50: (conservative Cx43 variant)
RPRPEDLEI
SEQ ID 51: (conservative Cx43 variant)
SSRASSRASSRPRPEDLEI,
SEQ ID 52: (conservative Cx45 variant)
GDGKNSVWV
SEQ ID 53: (conservative Cx45 variant)
SKAGSNKSTASSKSGDGKNSVWV
SEQ ID 54: (conservative Cx37 variant)
GQKPPSRPSSSASKKLYV
SEQ ID NO: 55 (non-active control peptide)
RQPKIWFPNRRKPWKIELDDPRPR
SEQ ID NO: 56 (HIV-Tat/ACT 1)
GRKKRRQRPPQ RPRPDDLEI
SEQ ID NO: 57 (Penetratin/ACT 1)
RQIKIWFQNRRMKWKK RPRPDDLEI
SEQ ID NO: 58 (Antp-3A/ACT 1)
RQIAIWFQNRRMKWAA RPRPDDLEI
SEQ ID NQ: 59 (Tat/ACT 1)
RKKRRQRRR RPRPDDLEI
SEQ ID NO: 60 (Buforin II/ACT 1)
TRSSRAGLQFPVGRVHRLLRK RPRPDDLEI
SEQ ID NO: 61 (Transportan/ACT 1)
GWTLNSAGYLLGKINKALAALAKKIL RPRPDDLEI
SEQ ID NO: 62 (MAP/ACT 1)
KLALKLALKALKAALKLA RPRPDDLEI
SEQ ID NO: 63 (K-FGF/ACT 1)
AAVALLPAVLLALLAP RPRPDDLEI
SEQ ID NO: 64 (Ku70/ACT 1)
VPMLKPMLKE RPRPDDLEI
SEQ ID NO: 65(Prion/ACT 1)
MANLGYWLLALFVTMWTDVGLCKKRPKP RPRPDDLEI
SEQ ID NO: 66 (pVEC/ACT 1)
LLIILRRRIRKQAHAHSK RPRPDDLEI
SEQ ID NO: 67 (Pep-11 ACT 1)
KETWWETWWTEWSQPKKKRKV RPRPDDLEI
SEQ ID NO: 68 (SynB1/ACT 1)
RGGRLSYSRRRFSTSTGR RPRPDDLEI
SEQ ID NO: 69 (Pep-7/ACT 1)
SDLWEMMMVSLACQY RPRPDDLEI
SEQ ID NO: 70 (HN-1/ACT 1)
TSPLNIHNGQKL RPRPDDLEI
SEQ ID NO: 71 (20 to 120 residues flanking amino
acid 363 of human Cx43)
KGKSDPYHATSGALSPAKDCGSQKYAYFNGCSSPTAPLSPMSPPGYKLVT
GDRNNSSCRNYNKQASEQNWANYSAEQNRMGQAGSTISNSHAQPFDFPDD
NQNSKKLAAGHELQPLAIVDQR
SEQ ID NO: 72 (20 to 120 residues flanking amino
acid 362 of chick Cx43)
KTDPYSHSGTMSPSKDCGSPKYAYYNGCSSPTAPLSPMSPPGYKLVTGDR
NNSSCRNYNKQASEQNWANYSAEQNRMGQAGSTISNSHAQPFDFADEHQN
TKKLASGHELQPLTIVDQRP
SEQ ID NO: 73 (20 to 120 residues flanking amino
acid 377 of human Cx45)
LGFGTIRDSLNSKRRELEDPGAYNYPFTWNTPSAPPGYNIAVKPDQIQYT
ELSNAKIAYKQNKANTAQEQQYGSHEENLPADLEALQREIRMAQERLDLA
VQAYSHQNNPHGPREKKAKV
SEQ ID NO: 74 (20 to 120 residues flanking amino
acid 375 of chick Cx45)
GFGTIRDTLNNKRKELEDSGTYNYPFTWNTPSAPPGYNIAVKPDQMQYT
ELSNAKMAYKQNKANIAQEQQYGSNEENIPADLENLQREIKVAQERLDM
AIQAYNNQNNPGSSSREKKSKA.
SEQ ID NO: 75 (20 to 120 residues flanking amino
acid 313 of human Cx37)
PYLVDCFVSRPTEKTIFIIFMLVVGLISLVLNLLELVHLLCRCLSRGMR
ARQGQDAPPTQGTSSDPYTDQVFFYLPVGQGPSSPPCPTYNGLSSSEQN
WANLTTEERLASSRPPLFLDPP
SEQ ID NO: 76 (20 to 120 residues flanking amino
acid 258 of rat Cx33)
CGSKEHGNRKMRGRLLLTYMASIFFKSVFEVAFLLIQWYLYGFTLSAVY
ICEQSPCPHRVDCFLSRPTEKTIFILFMLVVSMVSFVLNVIELFYVLFK
AIKNHLGNEKEEVYCNPVELQK.
SEQ ID NO: 77 (enhanced green fluorescent protein)
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSALSKDPNEKRDHMVLLEFVTAAGITLGMDELYK
SEQ ID NO: 78 (ACT 2)
CCCTCCTCCCGGGCCTCCTCCCGGGCCTCCTCCCGGCCCCGGCCCGAC
GACCTGGAGATC
SEQ ID NO: 79 (Cx43 portion of ACT1)
CGGCCCCGGCCCGACGACCTGGAGATC
SEQ ID NO: 80 (ACT 3)
CGGCCCCGGCCCGACGACCTGGAGGTG
SEQ ID NO: 81 (ACT 4)
CGGCCCCGGCCCGACGACGTGCCCGTG
SEQ ID NO: 82 (ACT 5)
AAGGCCCGGTCCGACGACCTGTCCGTG
SEQ ID NO: 83 (Antp)
CGGCAGCCCAAGATCTGGTTCCCCAACCGGCGGAAGCCCTGGAAG AAG
SEQ ID NO: 84 (Antp/ACT 2)
CGGCAGCCCAAGATCTGGTTCCCCAACCGGCGGAAGCCCTGGAAG
AAGCCCTCCTCCCGGGCCTCCTCCCGGGCCTCCTCCCGGCCCCGGCCC
GACGACCTGGAGATC
SEQ ID NO: 85 (ACT 1)
CGGCAGCCCAAGATCTGGTTCCCCAACCGGCGGAAGCCCTGGAAGAAGC
GGCCCCGGCCCGACGACCTGGAGATC
SEQ ID NO: 86 (Antp/ACT 3)
CGGCAGCCCAAGATCTGGTTCCCCAACCGGCGGAAGCCCTGGAAGAAGC
GGCCCCGGCCCGACGACCTGGAGGTG
SEQ ID NO: 87 (Antp/ACT 4)
CGGCAGCCCAAGATCTGGTTCCCCAACCGGCGGAAGCCCTGGAAGAAGC
GGCCCCGGCCCGACGACGTGCCCGTG
SEQ ID NO: 88 (Antp/ACT 5)
CGGCAGCCCAAGATCTGGTTCCCCAACCGGCGGAAGCCCTGGAAGAAGA
AGGCCCGGTCCGACGACCTGTCCGTG
SEQ ID NQ: 89 (Zebrafish alpha Cx43)
PCSRASSRMSSRARPDDLDV
SEQ ID NO: 90 (Chick alpha Cx36)
PRVSVPNFGRTQSSDSAYV
SEQ ID NO: 91 (Zebrafish alpha Cx36)
PRIVISMPNFGRTQSSDSAYV
SEQ ID NO: 92 (Cx43 isoleucine deletion)
RQPKIWFPNRRKPWKKRASSRASSRPRPDDLE

What is claimed is:

1. A method of preserving an organ or tissue for organ transplantation comprising contacting the organ with a solution comprising an isolated polypeptide comprising the carboxy-terminal amino acid sequence of an alpha connexin, or a conservative variant thereof.

2. The method of claim 1, wherein the organ is selected from the group consisting of heart, kidneys, liver, lungs, pancreas, intestine, and thymus.

3. The method of claim 1, wherein the polypeptide inhibits cellular injury in the organ or tissue.

4. The method of claim 1, wherein the method reverses cellular injury in the organ or tissue.

5. The method of claim 1, wherein the method rescues a marginal organ or tissue for transplantation, wherein the marginal organ or tissue would not otherwise be suitable for transplantation.

6. The method of claim 1, wherein the polypeptide
a. promotes cell-cell communication in the organ,
b. stabilizes gap junctions in cells of the organ,
c. stabilizes tight junctions in cells of the organ,
d. inhibits or mitigates hemichannel activity in cells of the organ,
e. inhibits apoptosis in cells of the organ,
f. inhibits mitochondrial oxidant production in cells of the organ,
g. promotes the integrity of endothelial cells of the organ,
h. promotes barrier function of endothelial cells of the organ, and/or
i. inhibits pro-inflammatory cytokine release from cells in the organ.

7. The method of claim 6, wherein the polypeptide inhibits pro-inflammatory cytokine release from cells in the organ, and wherein the pro-inflammatory cytokine is IL-8.

8. The method of claim 3, wherein the cellular injury is caused by cold preservation induced damage.

9. The method of claim 3, wherein the cellular injury is caused by hypoxia.

10. The method of claim 3, wherein the cellular injury is ischemia reperfusion injury (IRI).

11. The method of claim 3, wherein the cellular injury is ischemic reperfusion induced graft injury.

12. The method of claim 1, wherein the polypeptide comprises the amino acid sequence selected from the group consisting of SEQ ID NO: 1, SEQ ID NO: 2, SEQ ID NO: 3, SEQ ID NO: 4, and SEQ ID NO: 5.

13. The method of claim 13, wherein the polypeptide comprises the amino acid sequence of SEQ ID NO: 2.

14. The method of claim 1, wherein the polypeptide comprises an amino acid sequence with at least 65% sequence identity to the c-terminal most 9 amino acids of SEQ ID NO: 1.

15. The method of claim 1, wherein the polypeptide comprises from 4 to 30 contiguous amino acids of the carboxy-terminus of the alpha connexin.

16. The method of claim 1, wherein the alpha connexin is connexin 43.

17. The method of claim 1, wherein the isolated peptide comprises a cellular penetration sequence.

18. The method of claim 17, wherein the isolated peptide comprises SEQ ID NO: 9.

19. The method of claim 1, wherein the polypeptide is present in the solution at a concentration of about 1 μM to about 10 μM.

20. The method of claim 1, wherein the polypeptide is present in the solution at a concentration of less than about 10 μM.

21. The method of claim 1, wherein the polypeptide is present in the solution at a concentration of about 1 μM.

22. The method of claim 1, wherein the organ or tissue is contacted with the solution via ex vivo incubation, perfusion, or nebulization.

23. An organ or tissue preservation solution comprising a polypeptide comprising the carboxy-terminal amino acid sequence of an alpha connexin, or a conservative variant thereof, wherein the polypeptide is present in the solution in an amount effective for reversing cellular injury in the organ or tissue.

24. The organ preservation solution of claim 23, wherein the polypeptide comprises a sequence selected from the group consisting of SEQ ID NOs: 1, 2, 3, 4, 5, and 9.

25. The organ preservation solution of claim 23, wherein the polypeptide is present in the solution at a concentration of about 1 μM to about 10 μM.

26. The organ preservation solution of claim 23, wherein the polypeptide is present in the solution at a concentration of less than about 10 μM.

27. The organ preservation solution of claim 23, wherein the polypeptide is present in the solution at a concentration of about 1 μM.

28. A composition comprising (i) one or more organs or tissues for transplant and (ii) a polypeptide comprising the carboxy-terminal amino acid sequence of an alpha connexin, or a conservative variant thereof, wherein the polypeptide is present in the composition in an amount of about 1 μM to about 10 μM.

29. The composition of claim 28, wherein the polypeptide is present in the composition in an amount of less than about 10 μM.

30. The composition of claim 28, wherein the polypeptide is present in the composition in an amount of about 1 μM.

31. The composition of claim 28, wherein the polypeptide comprises a sequence selected from the group consisting of SEQ ID NOs: 1, 2, 3, 4, and 5.

32. The composition of claim 28, wherein the organ is selected from the group consisting of heart, kidneys, liver, lungs, pancreas, intestine, and thymus.
